(19) United States
(12) Patent Application Publication
(10) Pub. No.: US 2025/0387512 A1
(43) Pub. Date: Dec. 25, 2025

(54) METHODS OF IMPROVING SYSTEMIC DISEASE OUTCOMES BY INHIBITION OF ZHX2

(71) Applicant: RUSH UNIVERSITY MEDICAL CENTER, Chicago, IL (US)

(72) Inventor: Sumant Singh Chugh, Chicago, IL (US)

(73) Assignee: RUSH UNIVERSITY MEDICAL CENTER, Chicago, IL (US)

(21) Appl. No.: 18/703,666

(22) PCT Filed: Oct. 20, 2022
(86) PCT No.: PCT/US2022/047263

§ 371 Date: Apr. 22, 2024

Related U.S. Application Data
(60) Provisional application No. 63/272,404, filed on Oct. 27, 2021.

(51) Int. Cl. A61K 48/00; A61P 37/06; C07K 16/18; C12N 9/22; C12N 15/11; C12N 15/113; C12N 15/86

(52) U.S. Cl. CPC A61K 48/005; A61P 37/06; C07K 16/18; C12N 9/226; C12N 15/111; C12N 15/113; C12N 15/86; C07K 2317/76; C12N 2310/14; C12N 2310/20; C12N 2310/531; C12N 2740/15043; C12N 2750/14143

(57) ABSTRACT

The current invention provides novel approaches to the treatment of various disease states that lead to altered cytokine release or cytokine storms. These diseases include viral infections immunological and other non-immunological diseases. Specifically, the invention provides methods targeting ZHX2 including methods of inhibition, blocking or depletion of ZHX2 in order to treat various disease states.

### REFERENCE TO GOVERNMENT GRANTS

This invention was made with government support under R01 DK129522. R01 DK128203. R01 DK109713, and R01 DK111102 awarded by the National Institutes of Health. The government has certain rights in the invention.

### SEQUENCE LISTING

An electronic sequence listing (823089-00033.xml; size 36.0 KB; date of creation Jun. 10, 2025) submitted herewith is incorporated by reference in its entirety.

### FIELD

The general field of the present disclosure are novel approaches to the treatment of various disease states that lead to altered cytokine release or cytokine storms. These diseases include viral and non-viral infections, immunological and other non-immunological diseases.

### BACKGROUND

The Zinc Finger and Homeoboxes (ZHX) family transcriptional factors (ZHX1, ZHX2 and ZHX3) are expressed in multiple organs in the body and regulate a variety of the structurally and functionally important genes. See Macé et al “The Zinc Fingers and Homeoboxes 2 protein ZHX2 and its interacting proteins regulate upstream pathways in podocyte diseases,” (2020) Kidney Int 97 (4): pp. 753-764; Kawata H et al., “The mouse zinc-fingers and homeoboxes (ZHX) family; ZHX2 forms a heterodimer with ZHX3,” (2003) Gene. 323: pp 133-140. In the liver, ZHX2 is the major transcriptional repressor of α-fetoprotein expression in adult mice. BALB/cJ mice, but not 25 other mouse strains studied, have a mouse endogenous retrovirus in the first intron of the ZHX2 gene, which results in a predominantly non-functional transcript, causing ZHX2 downregulation and high α-fetoprotein protein levels in adult BALB/cJ mice. See Perincheri et al, “Hereditary persistence of alpha-fetoprotein and H19 expression in liver of BALB/cJ mice is due to a retrovirus insertion in the ZHX2 gene,” (2005) Proc Natl Acad Sci USA 102: pp. 396-401. In the kidney podocyte, ZHX2 is one of the most potent transcriptional repressors of WTI which makes it highly likely to be involved in the pathogenesis of FSGS (Macé et al 2020). In the kidney, podocytes express ZHX proteins mostly at the cell membrane, whereas in tubular cells, like many other organs, ZHX protein expression is predominantly nuclear (Macé et al 2020). In the right combinations and the setting of an altered ZHX2 expression state, systemic cytokine release could potentially induce migration of ZHX proteins from normal (Aminopeptidase A/APA, Ephrin B1) or putative alternative cell membrane anchors into the podocyte nucleus. Human and experimental MCD and most forms of FSGS are associated with low podocyte expression of transcriptional factor ZHX2. See Macé et al., “ZHX2 and its interacting proteins regulate upstream pathways in podocyte diseases,” (2020) Kidney Int. 97: pp. 753-764.

Viral infections trigger cytokine production as part of the innate and adaptive immune response. The inventors previously suspected that the extensive cytokine storm documented early in the pandemic may be involved in organ damage and developed novel evidence-based models of cytokine mediated end organ damage. See Huang et al. 2020. Of the three organs studied, the literature on cardiac involvement shows elevated cardiac Troponin I levels (that mimic an acute myocardial infarction), myocarditis, myocardial necrosis, pericarditis, arrythmias and heart failure. See Topo 2020; Inciardi et al., “Cardiac involvement in a patient with coronavirus disease 2019 (COVID-19),” (2020) JAMA Cardiol. 5: pp. 819-824. Evidence of liver injury include increased aminotransferase levels, hepatocyte injury, inflammation and steatosis. See Herta et al., “COVID-19 and the liver-Lessons learned,” (2021) Liver Int. 41 Suppl 1: pp. 1-8. Kidney manifestations are very common in hospitalized COVID-19 patients, with nearly 40% developing proteinuria, and about one-third developing acute kidney injury (AKI). See Cheng et al., “Kidney disease is associated with in-hospital death of patients with COVID-19,” (2020) Kidney Int. 97: pp. 829-838; Hirsch et al., “Acute kidney injury in patients hospitalized with COVID-19,” (2020) Kidney Int. 98: pp. 209-218. Kidney biopsy studies in COVID-19 patients with severe proteinuria and/or kidney dysfunction have most commonly documented the collapsing variant of focal and segmental glomerulosclerosis (FSGS) and acute kidney injury. See Kudose et al., “Kidney Biopsy Findings in Patients with COVID-19,” (2020) J Am Soc Nephrol. 31: pp. 1959-1968; Nasr et al., “Kidney Biopsy Findings in Patients With COVID-19, Kidney Injury and Proteinuria,” (2021) Am J Kidney Dis. 77: pp. 465-468 (2021). Despite suspicion of viral particles in early autopsy studies, kidney biopsies from living patients did not note any viral particles. See also Bradley et al., “Histopathology and ultrastructural findings of fatal COVID-19 infections in Washington State: a case series,” (2020) Lancet 396: pp. 320-332.

There are many other inflammatory and non-inflammatory diseases that affect cytokine release including non-respiratory viral infections, non-viral infections like bacterial, fungal or parasitic infections, immune-mediated disorders, cardiovascular pathology, diabetes, metabolic syndrome, neurodegeneration, and cancer, and aging.

However, little is known concerning the possible beneficial effects of ZHX2 inhibition on cytokine release and the treatment of various disease states. The present disclosure addresses these needs.

### SUMMARY

The current disclosure provides mechanisms targeting ZHX2. The disclosure describes methods of inhibition, blocking or depletion of ZHX2 in order to treat various disease states.

The inventors have previously demonstrated the how ZHX2 depletion in a ZHX2 hypomorph model, can affect cytokine storm related conditions. In initial studies, the inventors utilized ZHX2^{flox/flox} and NPHS2-promoter driven Cre mice. However, they subsequently used BALB/cJ mice, an established model of the ZHX2 hypomorph state, and BALB/c mice (ZHX2^{+/+}) to illustrate how ZHX2 expression affects cytokine storm related morbidity and mortality. See Macé et al. 2020; Perincheri et al., “Hereditary persistence of alpha-fetoprotein and H19 expression in liver of BALB/cJ mice is due to a retrovirus insertion in the ZHX2 gene.” (2005) Proc Natl Acad Sci USA 102: pp. 396-4011; Perincheri et al., “Characterization of the ETnII-alpha endogenous retroviral element in the BALB/cJ ZHX2 (Afr1) allele,” (2008) Mamm Genome 19: pp. 26-31; Gargalovic et al., “Quantitative trait locus mapping and identification of ZHX2 as a novel regulator of plasma lipid metabolism,” (2010) Circ Cardiovasc Genet. 3: pp. 60-67; Creasy et al., “Zinc Fingers and Homeoboxes 2 (ZHX2) Regulates Sexually Dimorphic Cyp Gene Expression in the Adult Mouse Liver,” (2016) Gene Expr. 17: pp. 7-17; Jiang et al., “ZHX2 (zinc fingers and homeoboxes) regulates major urinary protein gene expression in the mouse liver,” (2017) J Biol Chem 292: pp. 6765-6774 (2017); Erbilgin et al., “Transcription Factor ZHX2 Deficiency Reduces Atherosclerosis and Promotes Macrophage Apoptosis in Mice,” (2018) Arterioscler Thromb Vasc Biol. 38: pp. 2016-2027.

At low doses, COVID-19 cocktails, but not individual cytokines, induced glomerular injury and albuminuria in ZHX2 hypomorph and ZHX2+/+ mice to mimic COVID-19 related proteinuria. At high doses, COVID-19 cocktails, but not individual cytokines, induced common clinical manifestations of SARS-COV-2 disease, including acute heart injury, myocarditis, pericarditis, liver and kidney injury, and high mortality in ZHX2+/+ mice, whereas ZHX2 hypomorph mice were relatively protected. Activation of Signaling Transducer and Activator of Transcription 5 (STAT5), STAT6 and NFκB pathways in these organs was reduced and/or asynchronous in the ZHX2 hypomorph state. Using genome sequencing and CRISPR-Cas9, an insertion upstream of ZHX2 was identified as a cause of the human ZHX2 hypomorph state.

Thus, given that the glomerular expression of ZHX proteins, including ZHX2, is different (cell membrane associated in podocytes) from ZHX proteins in other organs and other parts of the kidney (predominantly nuclear), studies described in the current disclosure were undertaken to determine how the ZHX2 hypomorph state affects other organs and in other parts of the body in examples of disease.

In a general embodiment, methods of inhibiting, blocking or depleting ZHX2 in order to treat various disease states in a patient are provided.

In a general embodiment, methods of inhibiting, blocking or depleting ZHX2 can further include asynchronous activation of STAT5 or STAT6.

In some embodiments, ZHX2 can be inhibited, neutralized or depleted by the administration of and agent to the patient where the agent comprises an adeno-associated virus (AAV) or lentovirus-containing an a short-hairpin RNA (shRNA) against one ZXH2.

In some embodiments, the shRNA is commercially available and can be attached to or part of any vector known in the art including plasmids, viral vectors, bacteriophages, cosmids, and artificial chromosomes.

In other embodiments, the agent comprises a monoclonal antibody directed against the ZXH2. In yet other embodiments, the agent comprises a monoclonal antibody directed against ZXH2. In still other embodiments, the agent is an siRNA or antisense oligonucleotide that targets ZXH2.

In still other embodiments, the agent is a pharmacological agent that decreases ZHX2 expression.

In another embodiment, the agent is a pharmacological agent that reduces ZHX2 expression by binding or interacting directly or indirectly to its gene, or upstream or downstream of the ZHX2 gene.

In any embodiment, the disclosure provides methods of treating various disease states, by inhibiting, blocking or depleting ZHX2 where the disease states include inflammatory and non-inflammatory diseases that affect cytokine release including viral infections like SARS-Cov-1, SARS-Cov-2, other coronaviruses, influenza, parainfluenza, Respiratory Syncytial Virus, adenoviruses, enteroviruses, cytomegalovirus (CMV), Epstein Barr Virus (EBV), Middle East Respiratory Syndrome (MERS), and Ebola, non-respiratory viral infections, non-viral infections like bacterial, fungal and parasitic infections, immune-mediated disorders, cardiovascular pathology, diabetes, metabolic syndrome, organ transplantation, neurodegeneration, and cancer, and aging.

### BRIEF DESCRIPTION OF THE DRAWINGS

FIG. 1a-g depicts the development and characterization of COVID-19 cytokine storm models. (FIG. 1a) Schematic representation of COVID-19 induced cytokine storm in the context of human disease. (FIG. 1b) Composition of dose X of the COVID cocktails A to D. (FIG. 1c) Albuminuria after injecting different doses of Cocktail D into BALB/cJ mice (n=4 mice per group). X/2 is the threshold nephritogenic dose in BALB/cJ mice. (FIG. 1d) Albuminuria after injecting dose X of individual COVID cocktail components in BALB/cJ mice (n=4 mice per group). (FIG. 1e) Albuminuria after injecting COVID cocktails A to D dose X/2 in BALB/c mice (n=6 mice per group). (FIG. 1f) Albuminuria after injecting COVID cocktails A to D dose X/2 in BALB/cJ mice (n=6 mice per group). (FIG. 1g) Albuminuria after injecting BALB/c mice with intact Cocktail C dose X/2 or Cocktail C dose X/2 lacking individual components that target podocytes (n=6 mice per group). P<0.05; **P<0.01; ***P<0.001. All significant values are two-tail.

FIG. 2a-g depicts reduced systemic injury induced by high dose of Cocktail D (3×) in ZHX2 hypomorph BALB/cJ compared to ZHX2^{+/+} BALB/c mice. Dose 3× also has higher injury compared to lower doses or individual components at dose 3×. (FIG. 2a) Acute myocardial injury assessed by cardiac Troponin I levels (cTPI3) levels (n=8 mice per group). (FIG. 2b) Acute liver injury assessed by alanine aminotransferase (ALT) activity levels (n=8 mice per group). (FIG. 2c) Acute kidney injury assessed by serum creatinine measured using mass spectrometry (n=8 mice per group). (FIG. 2d) Histological characterization of acute cardiac injury (n=3 mice per group) using H&E-stained sections from Cocktail D dose 3× injected mice. Myocytolysis (red arrows), inflammation (black arrows), fibril disruption (blue arrows), hypereosinophilia (green arrows) and pericarditis (orange arrow) were noted. (FIG. 2e) Histological characterization of acute liver injury (n=3 mice per group) using H&E-stained sections from Cocktail D dose 3× injected mice. Hepatocellular injury (red arrows), inflammation (black arrows), prominent Kupfer cells (green arrows), regenerative changes (yellow arrows) and peri-central vein injury (blue arrow) were noted. (FIG. 2f) Histological assessment of acute kidney injury (n=3 mice per group) using PAS-dose 3× injected mice. Bottom three rows show proximal tubules, top row shows distal tubules. In proximal tubules, vacuolation (red arrows), brush border disruption (green arrows) and tubular degeneration (black arrows) were noted. In distal tubules, evidence of desquamation (blue arrows) was present. Foam cells were also noted (white arrows). Electron microscopy scale bars BALB/c, 2.66 μm; BALB/cJ, 2 μm. (FIG. 2g) Tables showing morphometric analysis and comparison of histological changes in the heart in BALB/c and BALB/cJ mice. (FIG. 2h) Tables showing morphometric analysis and comparison of histological changes in the liver in BALB/c and BALB/cJ mice. (FIG. 2i) Tables showing morphometric analysis and comparison of histological changes in the kidney in BALB/c and BALB/cJ mice. Light microscopy scale bars 20 μm. * P<0.05; **P<0.01; ***P<0.001, all values based on two-tail analysis.

FIG. 3 shows the cytokine storm induced mortality without and with depletion strategies for the effect of severe cytokine storms on systemic disease in ZHX2 hypomorph BALB/cJ mice. Number of mice injected per group are shown. The Control IgG group mortality of 1/6 or 16.6% in mice housed in metabolic cages reflects the mortality of the group without any antibody depletion strategy. All depleting antibodies or control IgG were injected intravenously one hour after model induction. Mortality is reduced after some depletion strategies.

FIG. 4 shows the cytokine storm induced mortality without and with depletion strategies for the effect of severe cytokine storms on systemic disease in ZHX2+/+BALB/c mice. Number of mice injected per group are shown in panel a. The Control IgG group mortality of 5/6 or 83.3% in mice housed in metabolic cages reflects the mortality of the group without any antibody depletion strategy. This is the number to be compared in terms of mortality in BALB/cJ mice in FIG. 3. The rest data in this table was generated by housing mice in normal cages, which was associated with a mortality of 2/6 or 33.3%. Under these conditions, timed urine collection for albuminuria was not conducted.

FIG. 5a-f: Genomic basis of the ZHX2 hypomorph state. Insertions and deletions (InDels) in non-coding DNA affect ZHX2 expression in patients with glomerular disease. (FIG. 5a) Schematic representation of ZHX2 and neighboring genes on chromosome 8. (FIG. 5b) Mapping of shared insertions and deletions among patients with MCD (n=9 patients), FSGS (n=19 patients), and COVID-19 related FSGS collapsing variant (n=8 patients). The most common shared insertion at 122,533,694 could be mapped at or close to the theoretical beginning of the rodent expressed gene S1c22a22 that is defunct in humans. Another shared insertion at 122,293,423 was located close to the theoretical end of S1c22a22. None of these InDels were noted in control subjects (n=33) or the 1000 Genomes project. Ex is Exon. (FIG. 5c) Tabular representation of shared insertions discussed above. (FIG. 5d) Schematic representation of CRISPR Cas9 assisted genome edited clones of a single cell derived cultured human podocyte cell line that contain an 8 bp insertion common between patients and a control subject (CRISPR A), or a 10 bp shared insertion at 122,533,694 that was absent in control subjects and the 1000 genomes project (CRISPR B). (FIG. 5e) Relative ZHX2 mRNA expression in above genome modified clones (CRISPR A, 3 clones, data pooled; study CRISPR B, 2 clones) compared to the parent single cell derived cultured human podocyte cell line (n=7 templates per clone). Dotted line represents expression in the parent cell line. (FIG. 5f) Western blot comparing ZHX2 expression in the Control single cell derived parent cell line and one of two mutant clones with insertion at 122,533,694. **P<0.01; ***P<0.001, all values based on two-tail analysis.

FIG. 6a-b depict the differences in mechanisms of COVID cocktail induced systemic injury (heart, liver and kidney) between in ZHX2 hypomorph BALB/cJ mice and ZHX2 BALB/c mice. Asynchronous activation of STAT5 and STAT6 signaling pathways in heart, liver and kidney by Cocktail D was noted in BALB/cJ compared to BALB/c mice. Cocktail D 3× dose injected mice (n=3 mice per group, each mouse organ assessed individually) were studied 15, 30 and 60 minutes after injection. Saline injected mice did not activate STAT pathway signaling, so data is not shown. (FIG. 6a) Graphical comparison of pSTAT5 Western blot densitometry expressed as a ratio with Lamin B1 (nuclear extracts) and STAT5 (cytosolic extracts) in heart, liver and kidney between BALB/c (normal ZHX2 expression) and BALB/cJ (low ZHX2 expression) mice. (FIG. 6b) Graphical comparison of pSTAT6 Western blot densitometry expressed as a ratio with Lamin B1 (nuclear extracts) and STAT6 (cytosolic extracts) in heart, liver and kidney between BALB/c and BALB/cJ mice. *P<0.05; **P<0.01; ***P<0.001, all values based on two-tail analysis.

FIG. 7a-b shows the mechanisms of COVID cytokine storm related glomerular injury. (FIG. 7a) Western blots to assess activation of pSTAT6 signaling in wild type and ZHX2 hypomorph (CRISPR B) cultured human podocytes incubated with human counterparts of Cocktail C cocktail (final concentration x/100,000; n=3 plates per condition). (FIG. 7b) Densitometry of Western blot of Cocktail C incubated wild type and CRISPR B podocytes from panel c. *P<0.05; **P<0.01; ***P<0.001, all values based on two-tail analysis.

FIG. 8a-e depicts examples of in vivo signaling mechanisms activated by COVID cocktails injected into mice. (FIG. 8a) Examples of NFκB/p-p65 (liver, 30 minutes), pSTAT6 (kidney 60 minutes) and pSTAT5 (heart, 15 minutes) activation by qualitative Western blot of whole organ protein extracts of mice (n=3 per group) injected with Cocktail D 3× or control saline. (FIG. 8b) Examples of nuclear extract purity studies by Western blot of nuclear and cytosol fractions with equal protein loading from heart, kidney and liver using anti-Lamin B1 antibody. Traces of Lamin B1 in the cytosol may be related to protein synthesis prior to transport to the nucleus. (FIG. 8c) Examples of assessment of GAPDH protein in heart cytosolic and nuclear fractions with equal protein loading from multiple mice. As expected, GAPDH is expressed in both fractions, with greater expression in cytosolic extracts. (FIG. 8d) Example of quantitative Western blots of BALB/c mouse heart nuclear protein extracts (20 μg protein per lane) in a Cocktail D (C′tail D) or control saline injection study assessed for pSTAT6 and Lamin B1 on separate blots developed on the same film. Abbreviated mouse numbers are shown for each lane. (FIG. 8e) Example of quantitative Western blots of BALB/cJ mouse heart cytosolic protein extracts (20 μg protein per lane) in a Cocktail D (C′tail D) or control saline injection study assessed for pSTAT5 and STAT5 on separate blots developed on the same film. Abbreviated mouse numbers are shown for each lane.

FIG. 9a-c: (FIG. 9a) Plasma IL-4Rα levels assessed by ELISA in general COVID-19 patients, age, sex and race matched healthy controls, and COVID-19 patients with proteinuria. Number of patient samples assayed is shown below. (FIG. 9b) Electron microscopy images of BALB/cJ mouse glomeruli on Day 1 after injection of Cocktail D dose X/2. Areas of focal foot process effacement (black arrows), endothelial vacuolation (green circles), and endothelial hypertrophy (blue circles) were noted. (FIG. 9c) Serum creatinine, assayed by Mass Spectrometry, is not increased in the COVID cytokine cocktail dose X/2 models (BALB/c and BALB/cJ mice; n=6 mice per group). Scale bars 0.5 μm.

FIG. 10a-o shows the characterization of ZHX2 hypomorph state in BALB/cJ mice: (FIG. 10a) Plasma creatine kinase, a marker of skeletal muscle injury, in BALB/cJ mice (n=4 mice per group) 24 hours after injection of Cocktail D at different doses. (FIG. 10b) Serum Cardiac Troponin I level data derived from FIG. 2a, plotted again for higher resolution of lesser increase in levels among some single cytokine injected groups. (FIG. 10c) Serum ALT level data derived from FIG. 2b, plotted again for higher resolution of lesser increase in levels among some single cytokine injected groups. (FIG. 10d) 18-hour albuminuria in BALB/cJ mice injected with Cocktail D 3× or single cytokines dose 3×, corresponding to FIGS. 2a-c. (FIG. 10e) 18-hour albuminuria in BALB/c mice injected with single cytokine dose 3×, corresponding to FIGS. 2a-c. Given their high mortality after Cocktail D 3×, metabolic cage housing for timed urine collection is not feasible in BALB/c mice. (FIG. 10f) Fold-difference in heart and skeletal muscle ZHX2 mRNA expression in BALB/cJ compared to BALB/c mice assessed by real time PCR (n=6 templates per group). Lower levels of ZHX2 mRNA expression in the liver were previously published and serve as a positive control for this phenomenon. Three-fold difference was taken as significant. (FIG. 10g) Real time PCR comparison of expression of cytokine receptors, Ace2, Zhx1, Zhx3, and signaling pathway proteins STAT5, STAT6 and P-65 (NFκB) between BALB/cJ and BALB/c mouse heart (n=6 templates per group). Three-fold difference was taken as significant. (FIG. 10h) Real time PCR comparison of expression of cytokine receptors, Ace2, Zhx1, Zhx3, and signaling pathway proteins STAT5, STAT6 and P-65 (NFκB) between BALB/cJ and BALB/c mouse liver (n=6 templates per group). Three-fold difference was taken as significant. (FIG. 10i) Real time PCR comparison of expression of cytokine receptors, Ace2, Zhx1, Zhx3, and signaling pathway proteins STAT5, STAT6 and P-65 (NFκB) between BALB/cJ and BALB/c mouse skeletal muscle (n=6 templates per group). Three-fold difference was taken as significant. & 3.06+0.40 fold higher expression of STAT5 in BALB/c mice. (FIG. 10j) Real time PCR comparison of expression of cytokine receptors, Ace2, and signaling pathway proteins STAT5, STAT6 and P-65 (NFκB) between BALB/cJ and BALB/c mouse glomeruli (n=6 templates per group). Three-fold difference was taken as significant. Other IL-2R chains are not expressed in mouse glomeruli, and Zhx1 and Zhx3 are previously published (Macé et al 2020). (FIG. 10k) Electron microscopy of BALB/cJ mouse kidney glomeruli 24 hours after injection Cocktail D dose 3×. Multifocal foot processes effacement (red arrows), endothelial hypertrophy (green arrows) and glomerular basement membrane (GBM) remodeling (blue arrows) were present. (FIG. 10l) Electron microscopy of BALB/c mouse kidney glomeruli 24 hours after injection Cocktail D dose 3×. Extensive foot processes effacement (red arrows), endothelial hypertrophy (green arrows) and glomerular basement membrane (GBM) remodeling (blue arrows) were present. (FIG. 10m) Hematoxylin and Eosin-stained skeletal muscle from BALB/cJ mice 24 hours after injection of Cocktail D dose 3×. Focal inflammation (black arrows) was noted in some sections. (FIG. 10n) Albuminuria after induction of the Cocktail D model in BALB/cJ mice (n=6 mice per group; dose X/2), followed by Control IgG or combinations of depleting antibodies one hour after model induction. (FIG. 100) Albuminuria after induction of Cocktail C in BALB/c mice (n=6 mice per group; dose X/2), followed by receptor blockage using antibodies against IL-4Rα, TNFR1 and IL-10Rβ, or control IgG. Scale bars (k) 0.5 μm, (1) 0.5 μm, (m) 20 μm. *P<0.05; **P<0.01; >**P<0.001.

FIG. 11a-c: (FIG. 11a) Single and shared Insertions and Deletions (InDels) in the study population (FIG. 11b) InDels in FSGS patients expanded by disease sub-categories. (FIG. 11c) Shared InDels in diabetic nephropathy patients (n=13).

FIG. 12a-c: (FIG. 12a) List of single InDels in the study population. (FIG. 12b) The S1c22a22 gene between Has2 and ZHX2 in rodents, and its absence in larger animals and humans, arranged by size and heart rate. (FIG. 12c) Ponceau Red stained membrane from blot shown in FIG. 5f.

FIG. 13a-c: (FIG. 13a) Confocal expression of cytokine receptors in BALB/c mouse glomeruli. White arrows indicate receptor expression in podocytes (P), endothelial (E) and mesangial (M) cells. Since TNFR1 is expressed in podocytes and endothelial cells, only partial co-localization with nephrin (blue), a podocyte protein, is noted. Green color is nuclear stain. (FIG. 13b) Confocal expression (red) of ACE-2 and cytokine receptors in BALB/c mouse kidney tubules. Most images show proximal tubules, except IL-10Rβ image is collecting duct. (FIG. 13c) Characterization of antibodies used for depletion studies using recombinant proteins that make up the cytokine cocktails. Scale bars (b) 20 μm (c) 20 μm.

### DETAILED DESCRIPTION

The current disclosure provides mechanisms targeting ZHX2. The disclosure describes methods of inhibition, blocking or depletion of ZHX2 in order to treat various disease states. The inventors contemplate that any of the disclosed methods of inhibiting, blocking or depleting ZHX2 can further include altering the activation of STAT5, STAT6 or NFκB.

Methods are disclosed in which ZHX2 can be inhibited, neutralized or depleted by the administration of an agent to the patient where the agent comprises an adeno-associated virus (AAV) or lentovirus-containing an a short-hairpin RNA (shRNA) against one ZXH2. The inventors contemplate that the shRNA may be commercially available and can be attached to or part of any vector known in the art including plasmids, viral vectors, bacteriophages, cosmids, and artificial chromosomes.

Methods are disclosed in which ZHX2 can be inhibited, neutralized or depleted by the administration of a polyclonal or a monoclonal antibody directed against the ZXH2.

Methods are disclosed in which ZHX2 can be inhibited, neutralized or depleted by the administration of an siRNA or antisense oligonucleotide that targets ZXH2.

Methods are disclosed in which ZHX2 can be inhibited, neutralized or depleted by the administration of a pharmacological agent that decreases ZHX2 expression.

Methods are disclosed in which ZHX2 can be inhibited, neutralized or depleted by a pharmacological agent that binds or interacts directly or indirectly with the ZHX2 gene, or upstream or downstream of the ZHX2 gene, and make reversible or irreversible changes at these sites.

It is also contemplated that ZHX2 can be silenced or “turned off” by a pharmacological agent that inhibits or blocks a protein that interacts with ZHX2 in the nucleus. Examples of such proteins include other ZHX proteins, Nuclear Factor Y-A and many others.

In some embodiments, the disclosure provides methods of treating various disease states, by inhibiting, blocking or depleting ZHX2 where the disease states include inflammatory and non-inflammatory diseases that affect cytokine release including viral infections like SARS-Cov-1, SARS-Cov-2, other coronaviruses, influenza, parainfluenza, Respiratory Syncytial Virus, adenoviruses, enteroviruses, cytomegalovirus (CMV), Epstein Barr Virus (EBV), Middle East Respiratory Syndrome (MERS), and Ebola, non-respiratory viral infections, non-viral infections like bacterial, fungal and parasitic infections, immune-mediated disorders, cardiovascular pathology, diabetes, metabolic syndrome, organ transplantation, neurodegeneration, and cancer, and aging.

Throughout this disclosure, various quantities, such as amounts, sizes, dimensions, proportions and the like, are presented in a range format. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human.

### ZHX2 Inhibitors

The current disclosure contemplates that ZHX2 can be neutralized or inhibited by several different non-limiting methods. For example, as described herein, ZHX2 can be neutralized or inhibited by administration of a therapeutically effective amount of an agent where the agent comprises an adeno-associated virus (AAV) or lentovirus-containing an a short-hairpin RNA (shRNA) against ZHX2 (sh-XHX2). In some embodiments, the sh-ZHX2 is commercially available and can be attached to or part of any vector known in the art including plasmids, viral vectors, bacteriophages, cosmids, and artificial chromosomes. Alternatively, as described herein, ZHX2 can be neutralized or inhibited by administration of a therapeutically effective amount of an agent where the agent comprises an antibody, bivalent antibody or a monoclonal antibody directed against the ZHX2. Further, as described herein, ZHX2 can be neutralized or inhibited by administration of a therapeutically effective amount of an agent where the agent comprises an siRNA or antisense oligonucleotide that targets ZHX2. Also, as contemplated herein, ZHX2 can be neutralized or inhibited by administration of a therapeutically effective amount of an agent where the agent is a pharmacological agent that can comprise an antagonist or an antagonist that binds to ZHX2-binding proteins or DNA sequences and prevents the binding of ZHX2. Also, as contemplated herein, a pharmacological agent can bind or interact directly or indirectly with the ZHX2 gene, or upstream or downstream of the ZHX2 gene. The ZHX2 inhibitors or a composition therein can be administered once per day, two or more times daily or once per week. The ZHX2 inhibitors or composition containing the same can occur by any conventional means including orally intramuscularly, intraperitoneally or intravenously into the subject. If injected, they can be injected at a single site per dose or multiple sites per dose.

### ZHX2 Antibodies and Related Inhibitors

More specifically a ZHX2 inhibitor is a polyclonal or monoclonal antibody directed against ZHX2. Examples of suitable antibodies directed against ZHX2 are disclosed herein and known to those of skill in the art. The ZHX2 antibody can also include an antibody fragment or a bivalent antibody or fragment thereof, inhibiting ZHX2. As described herein, the ZHX2 inhibitor may be part of a pharmaceutical composition where the composition may include either an antibody or fragment thereof for ZHX2.

The anti-ZHX2 antibodies described herein can be made or obtained by any means known in the art, including commercially. It is also contemplated that an antibody can be specifically reactive with ZHX2 or a particular ZHX2 polypeptide may also be used as an antagonist. An anti-ZHX2 herein may be an antibody or fragment thereof that binds to ZHX2 or a cytokine or a bivalent antibody that binds to ZHX2 and another suitable target.

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” and other interchangeable terms for similar binding fragments such as described below.

Native antibodies and native immunoglobulins are usually heterotetrameric glycoproteins of about 150,000 Daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}” or “VH”) followed by a number of constant domains (“C_{H}” or “CH”). Each light chain has a variable domain at one end (“V_{L}” or “VL”) and a constant domain (“C_{L}” or “CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The ZHX2 as described herein can be a “synthetic polypeptide” derived from a “synthetic polynucleotide” derived from a “synthetic gene,” meaning that the corresponding polynucleotide sequence or portion thereof, or amino acid sequence or portion thereof, is derived, from a sequence that has been designed, or synthesized de novo, or modified, compared to an equivalent naturally occurring sequence. Synthetic polynucleotides (antibodies or antigen binding fragments) or synthetic genes can be prepared by methods known in the art, including but not limited to, the chemical synthesis of nucleic acid or amino acid sequences. Synthetic genes are typically different from naturally occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

With respect to anti-ZHX2 antibodies, the term “antigen” refers to ZHX2 or any fragment of the protein molecules thereof.

The terms “antigen-binding portion of an antibody,” “antigen-binding fragment,” “antigen-binding domain,” “antibody fragment” or a “functional fragment of an antibody” are used interchangeably herein to refer to one or more fragments of an antibody that retain the ability to specifically bind to ZHX2.

It is contemplated that the ZHX2 antibodies may also include “diabodies” which refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. See for example, EP 404,097; WO 93/11161; and Hollinger et al., Proc. Natl. Acad. Sci. USA 90:6444 6448 (1993).

It is contemplated that the ZHX2 may also include “chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin are inserted in place of the murine Fc. See for example, Jones et al., Nature 321:522-525 (1986); Reichmann et al., Nature 332:323-329 (1988); and Presta, Curr. Op. Struct. Biol., 2:593-596 (1992).

It is contemplated that the ZHX2 antibodies may also include a “monoclonal antibody” which refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which can include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, monoclonal antibodies can be made by a hybridoma method, recombinant DNA methods, or isolated from phage antibody.

As used herein, “immunoreactive” refers to binding agents, antibodies or fragments thereof that are specific to a sequence of amino acid residues on ZHX2 (“binding site” or “epitope”), yet if are cross-reactive to other peptides/proteins, are not toxic at the levels at which they are formulated for administration to human use. The term “binding” refers to a direct association between two molecules, due to, for example, covalent, electrostatic, hydrophobic, and ionic and/or hydrogen-bond interactions under physiological conditions and including interactions such as salt bridges and water bridges and any other conventional binding means. The term “preferentially binds” means that the binding agent binds to the binding site with greater affinity than it binds unrelated amino acid sequences.

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

“Epitope” refers to that portion of an antigen or other macromolecule capable of forming a binding interaction with the variable region binding pocket of an antibody.

The term “specific” refers to a situation in which an antibody will not show any significant binding to molecules other than the antigen containing the epitope recognized by the antibody. The term is also applicable where, for example, an antigen binding domain is specific for a particular epitope which is carried by a number of antigens, in which case the antibody will be able to bind to the various antigens carrying the epitope. The terms “preferentially binds” or “specifically binds” mean that the antibodies bind to an epitope with greater affinity than it binds unrelated amino acid sequences, and, if cross-reactive to other polypeptides containing the epitope, are not toxic at the levels at which they are formulated for administration to human use.

The term “binding” refers to a direct association between two molecules, due to, for example, covalent, electrostatic, hydrophobic, and ionic and/or hydrogen-bond interactions under physiological conditions and includes interactions such as salt bridges and water bridges, as well as any other conventional means of binding.

As contemplated herein, a ZHX2 inhibitor may be generated through gene expression technology. The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

It is also contemplated that ZHX2 can be silenced or “turned” off” through the use of CRISPR technology as disclosed herein in the Examples.

It is also contemplated that ZHX2 can be silenced or “turned off” by a pharmacological agent can binds or interacts directly or indirectly with the ZHX2 gene, or upstream or downstream of the ZHX2 gene, or makes reversible or irreversible changes at these sites.

It is also contemplated that ZHX2 can be silenced or “turned off” by a pharmacological agent that inhibits or blocks a protein that interacts with ZHX2 in the nucleus. Examples of such proteins include other ZHX proteins, Nuclear Factor Y-A and many others.

### General Methods

### COVID Cytokine Cocktails, and Related Animal Studies

All animal studies conducted were approved by the IACUC at Rush University or the University of Alabama at Birmingham. All animals received humane treatment per protocol. Methods for Dynabead assisted mouse glomerular isolation, rat glomerular isolation by sieving, histological section tissue preservation, timed 18-hour urine collection in metabolic cages in the absence of food, assessment of albuminuria and proteinuria, real time PCR, confocal imaging, electron microscopy and sample processing, histology for light microscopy, Western blot and co-immunoprecipitation are previously described and known. The following were assayed using commercially available kits using serum samples; mouse ALT (BioVision K752-100), mouse cardiac Troponin I Type 3 (Novus Biologicals NBP3-00456), mouse Creatine Kinase (Abcam ab 155901) and human IL-4Rα ELISA (Abcam ab46022). The following antibodies were purchased for Western blot: anti-pSTAT6 (Cell Signaling Technology, Inc. Danvers MA, USA; cat #56554, 1:500 dilution); anti-STAT6 (Cell Signaling Technology, Inc. Cat #5397, 1:500 dilution). Antibodies against ZHX1, ZHX2 and ZHX3 are previously described. Maće et al. 2020; Liu et al., “ZHX proteins regulate podocyte gene expression during the development of nephrotic syndrome,” (2006) J. Biol. Chem. 281: pp. 39681-39692; Clement et al., “Early changes in gene expression that influence the course of primary glomerular disease,” (2007) Kidney Int. 72: pp. 337-347.

All cytokines, soluble receptors, and antibodies were injected intravenously in rodents, and are listed below:

| | | | | Injected |
|---|---|---|---|---|
| Cytokine/Receptor | Rat | Mouse | Human | Antibody |
| IL-2 | + | + | + | + |
| IL-4Rα | + | + | + | + |
| IL-4 | + | + | + | + |
| IL-13 | + | + | N.A. | + |
| IL-6 | + | + | + | + |
| IL-10 | + | + | + | + |
| Interferon-γ | + | + | + | + |
| TNF-α | + | + | + | + |
| ACE-2 | + | + | + | + |
| ICAM-1 | + | + | + | N.A. |
| TNFRI | | | | + |
| IL-10Rβ | | | | + |
| Rat gamma globulin | | | | + |
| Syrian hamster | | | | + |
| gamma globulin | | | | |

Antibodies used for depletion studies were characterized by Western blot using the corresponding recombinant protein. Each dose of cytokine cocktail was dissolved in a final volume of 100 μL of sterile 0.9% saline. BALB/cJ (Jackson Labs) and BALB/c (Envigo) mice were purchased at age 8 weeks, acclimatized for 2 weeks, and baseline 18-hour urine collection and tail blood sampling conducted. An extra baseline urine collection was conducted for BALB/cJ mice. Most in vivo studies were conducted between age 10 and 15 weeks. The nephritogenic dose spectrum of cytokine cocktails was established for BALB/cJ, BALB/c, IL4r^{-/-} (Jackson Labs), ZHX2^{flox/flox}, NPHS2^{cre/cre} During mouse cytokine studies using threshold nephritogenic doses (BALB/cJ, BALB/c, Il4r^{-/-} in BALB/cJ background, X/2; ZHX2^{flox/flox}; NPHS2^{cre/cre}, X/15), 100 μL of 0.9% saline was given intraperitoneally immediately after the intravenous cytokine cocktail dose to maintain intravascular hydration. Two additional intraperitoneal injection of 100 μL of 0.9% saline were given at 6 and 23 hours in the intermediate and high cocktail models. During cytokine depletion studies, different groups of mice received 50 μg of control IgG or the respective antibody or antibody combination intravenously 1 hour after the administration of the mouse cytokine cocktail.

### Mass Spectrometry Assay for Plasma Creatinine

Serum creatinine was measured by LC/MS/MS using an Agilent 1290 Infinity II LC system in combination with a 2×50 mm, 2 μm Tosoh Bioscience TSK-GEL amide-80 LC column, interfaced to an Agilent 6495 Triple Quadrupole. The oven temperature was fixed at 40° C. The mobile phase consisted of 10 mM ammonium acetate in LCMS-grade water (35%) and LCMS-grade acetonitrile (ACN; 65%). Synthetic creatinine (ranging from 20 μg/ml to 0.16 ug/ml; Sigma) and isotope-labeled creatinine (D3-creatinine, 10 μg/ml; Sigma) were used as standard and internal standard, respectively. Then, 10 μl of sample or standard was combined with 5 μl internal standard and 235 ul 100% ACN, vortexed and centrifuged at 4° C. for 15 min at 15000 rpm. The supernatant was transferred to a new tube with 200 ul 10 mM ammonium acetate and 65% acetonitrile in LCMS-grade water, vortexed, centrifuged at 4° C. for 15 min at 15000 rpm and subsequently measured. All samples were measured in duplicate.

### Sources of Human Genomic DNA and Human Kidney Biopsies

Genomic DNA samples from 36 patients with nephrotic syndrome, 33 control subjects, and 16 patients with diabetic nephropathy were obtained from the following sources (a) immortalized monocytes from plasma of nephrotic syndrome patients at the University of Alabama at Birmingham obtained via an IRB approved protocol X080813001 for collecting DNA, blood and urine samples. (b) IRB approved study at the Instituto Nacional De Cardiologia in Mexico City (CONACYT 34751M, CONACYT 11-05, and DPAGA-UNAM IN-201902) that included archived kidney biopsies from patients with glomerular diseases or pre-implantation kidney biopsies from healthy living related kidney donors. (c) Archived kidney biopsy, IRB exempt, from Hospital Nacional Alberto Sabogal Essalud, Lima, Peru. (d) Archived human DNA of previously published FSGS patients 43, 44 from the Duke Molecular Physiology Institute with known mutations in podocyte expressed related genes. (e) Coriell Cell Repositories, that archive DNA from the 1000 Genomes Project and the HAPMAP Project. For analytical comparisons between cases and controls, the 1000 genomes project phase 3 Ensambl v84 was included as a single additional control.

### Agilent Custom Capture and High Throughput Illumina Sequencing

A custom capture sequencing panel was created to isolate the genomic interval between HAS2 and ZHX2 on Chromosome 8. The target interval was uploaded to the SureDesign website for Agilent SureSelect capture probe design and synthesis (Agilent Technologies, Santa Clara CA). Genomic DNA library preparation and interval capture was done using the QXT SureSelect kit as per the manufacturer's instructions (Agilent Technologies). The resulting DNA libraries were quantitated by QPCR (Kapa Biosystems, Wilmington MA) and sequenced on the Illumina HiSeq 2500 or NextSeq 500 with paired end 100 bp sequencing following standard protocols. Approximately 15 million sequences were obtained per reaction. FASTQ file generation was done using bcl2fastq converter from Illumina (Illumina, Inc., San Diego CA). Paired Illumina sequences compared with hg38 database (GRCh38.p13 Primary Assembly) using CLC Genomics software (Version 12, Qiagen, Venlo, the Netherlands). Insertion and deletions of 3 bp size or larger and a minimum of 20 sequence reads were selected for analysis. Fisher test comparison of insertions and deletions in study and control subjects was exported in Excel format, followed by software assisted and manual exclusion of all insertions and deletions present in controls. Only insertions and deletions that were subsequently confirmed using IGV browser software (Broad Institute, Boston MA) were included. Establishment of homozygosity required presence of the InDel in over 85% of sequences, and subsequent confirmation by IGV. Minor discrepancies (1-2 base pair position differences) in the site of the insertion or deletion were occasionally noted between the two software and were resolved by Sanger sequencing while designing CRISPR Cas9 studies. All genomic numbering is based on hg38 and CLC genomics software.

### Hypothetical Projection of S1c22a22 Location in the Human Genome

BLAST based margins used the two peripheral parts of the mouse gene that matched with the human genome. BLAST and size-based projections extended the BLAST based margins to the size of the mouse gene at either end.

### Genome Editing in Cultured Human Podocytes Using CRISPR/Cas9

The basic methodology for CRISPR Cas9 is previously published. See Cong et al., “Multiplex Genome Engineering using CRISPR/Cas Systems,” (2013) Science 339: pp. 819-823. A single cell derived clone of cells was generated from an established early passage immortalized human podocyte cell line 51 and used for genome editing studies. The oligonucleotides and primers used are listed in Table 4.

TABLE 4

| Name | Application | Primer/ Probe Sequence |
|---|---|---|
| | Generation of Crispr-cas9 | |
| | mutants | |
| | CRISPR B | |
| G0016 | SgRNA generation | 5′-CAC CGA CTG GTA AAC CAC TTA GGG C-3′ |
| G0017 | SgRNA generation | 5′-AAA CGC CCT AAG TGG TTT ACC AGT C-3′ |
| K1145 | SgRNA plasmid sequencing | 5′-GCA TAT ACG ATA CAA GGC TGT TAG AGA G-3′ |
| K1195 | Donor plasmid | 5′-CGG GCC GGA TCC CTA GAT GTA GCA TTA CCA GGG TGG-3′ |
| K1196 | Donor plasmid | 5′-GGC CGA AGC TTG CAG AGA AGA TCA CGA TAG ATT AGA |
| | | AGA TG-3′ |
| K1207 | Sequencing of donor plasmid | 5′-GGT TTC CTT GTT ATA TCA CCA G-3′ |
| K1215 | Quickchange Mutagenesis | 5′-GCT CTA GGA TGA CTG GTA AAC CAC TTA GGG CAG TCG TCC |
| | | CCA GAC CTG GTC TGT GGC CTG TTA G-3′ |
| K1216 | Quickchange Mutagenesis | 5′-CTA ACA GGC CAC AGA CCA GGT CTG GGG ACG ACT GCC CTA |
| | | AGT GGT TTA CCA GTC ATC CTA GAG C-3′ |
| K1219 | Plasmid linear amplification | 5′-GAT TAT CTT TCT AGG GTT AAC GAA CTT CAA GTA ATC AAG |
| | | AGC AGC-3′ |
| K1220 | Plasmid linear amplification | 5′-CGC AGA CTA TCT TTC TAG GGT TAA CTT TGT AGA ATG CTT |
| | | CTC G-3′ |
| K1217 | Puromycin cassette | 5′-CGA GAA GCA TTC TAC AAA GTT AAC CCT AGA AAG ATA GTC |
| | | TGC G-3′ |
| K1218 | Puromycin cassette | 5′-GCT GCT CTT GAT TAC TTG AAG TTC GTT AAC CCT AGA AAG |
| | | ATA ATC-3′ |
| K1189 | Genome editing | 5′-ACA CTG ACG ACA TGG TTC |
| | | TAC AGT CTC TGA AAC ATA GAA |
| | | GGC AC-3′ |
| K1188 | Genome editing | 5′-TAC GGT AGC AGA GAC TTG GTC TGA GAA TCT AAT ACC GCT |
| | | GAT CTG-3′ |
| | CRISPR A | |
| G0003 | SgRNA generation | 5′-CAC CGA CCC ATC CAT ACA CTT ACC C-3′ |
| G0004 | SgRNA generation | 5′-AAA CGG GTA AGT GTA TGG ATG GGT C-3′ |
| K1145 | SgRNA plasmid sequencing | 5′-GCA TAT ACG ATA CAA GGC TGT TAG AGA G-3′ |
| K1140 | Donor plasmid | 5′-GGC GGC ACT AGT CTA GCT GGC TTG ACT TTA CAA GAC GAT |
| | | TCC ATC C-3′ |
| K1141 | Donor plasmid | 5′-GGG CGG ATC CCT GCA CTC AGT ATT CTG CAA GTC CTG TAG |
| | | C-3′ |
| K1151 | Sequencing of donor plasmid | 5′-CGA TCT CCT GAC CTC AAG-3′ |
| | | 5′-GTG CCT GGC CTG TTA TGA TCT TCT TAC TCA TTT GAT AGC |
| | | ACC AGT GTC CTG AGA AAA ATA ACA TAT ACT |
| K1149 | Quickchange Mutagenesis | CCA TTA CCC ATC CAT ACA CTT ACC CAG GCA CTC ATT CAC CAT |
| | | ATT AAC TAG ATA GAC ACA TGA TGT TGC |
| | | TGC TCC TGT TGA TGA TAA CAA TGT TGA GG-3′ |
| | | 5′-CCT CAA CAT TGT TAT CAT CAA CAG GAG CAG CAA CAT CAT |
| | | GTG TCT ATC TAG TTA ATA TGG TGA ATG AGT |
| K1150 | Quickchange Mutagenesis | GAC TGG GTA AGT GTA TGG ATG GGT AAT GGA GTA TAT GTT ATT |
| | | TTT CTC AGG ACA CTG GTG CTA TCA AAT |
| | | GAG TAA GAA GAT CAT AAC AGG CCA GGA C-3′ |
| K1163 | Plasmid linear amplification | 5′-CGT CAC AAT ATG ATT ATC TTT CTA GGG TTA ACT AGA TAG |
| | | ACA CAT GAT GTT GCT GCT CC-3′ |
| K1164 | Plasmid linear amplification | 5′-CGT CAA TTT TAC GCA GAC TAT CTT TCT AGG GTT AAT ATG |
| | | GTG AAT GAG TGA CTG GG-3′ |
| K1153 | Puromycin cassette | 5′-CCC AGT CAC TCA TTC ACC ATA TTA ACC CTA GAA AGA TAG |
| | | TCT GCG TAA AAT TGA CG-3′ |
| K1154 | Puromycin cassette | 5′-GGA GCA GCA ACA TCA TGT GTC TAT CTA GTT AAC CCT AGA |
| | | AAG ATA ATC ATA TTG TGA CG-3′ |
| K1138 | Genome editing | 5′-ACA CTG ACG ACA TGG TTC TAC AGT TAT GAT CTT CTT ACT |
| | | CAT TTG ATA GCA CCA GTG TCC-3′ |
| K1139 | Genome editing | 5′-TAC GGT AGC AGA GAC TTG GTC TGA AAG GAG CAG TGT TGA |
| | | TCT AGA GAG AGC C-3′ |
| | Real time PCR | |
| H826 | Human ZHX2 forward primer | CGGAACTGGCTGAATCAGACT |
| H827 | Human ZHX2 reverse primer | CAGCACAGCAGTTCTAACAGACTT |
| P246 | FAM-MGB Probe | TGCAGAGGCTGGCCA |

### CRISPR B

Generation of the sgRNA plasmid: In order to introduce a 10 bp insertion (CACACACACA), sgRNA recognizing a specific site 45 bp downstream of the insertion site (Chr8-122,533,694-122,533,695) was designed using the Benchling website (https://benchling.com). Oligos G0016 and G0017 were phosphorylated and annealed using T4 Polynucleotide Kinase (NEB), digested with Bbsl and ligated into pX330-U6-Chimeric_BB-CBh-hSpCas9 plasmid (a gift from Feng Zhang, Addgene plasmid #42230) using T7 DNA ligase (New England Biolabs). The ligation product was treated with PlasmidSafe exonuclease (Epicentre) to prevent unwanted recombination products and then transformed into One Shot TOP10 cells (Invitrogen). Ten colonies were picked up and plasmids were isolated using QIAprep Spin Miniprep Kit (QIAgen). Plasmid DNA was sequenced using primer K1145 (see Table 4).

Generation of the donor plasmid: The human genomic sequence from patient E58-13 containing the insertion under study was amplified using KAPA HiFi HotStart PCR Kit (Kapa Biosystems), the specific patient genomic DNA and primers K1195 and K1196, and cloned into pBlueScript II KS+ vector between the BamHI and HindIII restriction sites. Plasmid DNA was sequenced using K1207 to confirm the presence of the insertion. A single mutation in the PAM sequence was made to prevent cutting of this donor template plasmid using Quikchange mutagenesis kit (Agilent Technologies) and primers K1215 and K1216, and the change confirmed by sequencing. Next, this plasmid was amplified in linear fashion using primers K1219 and K1220, and the PCR product digested with DpnI to remove any residual circular template plasmid. The antibiotic selection cassette (Puromycin resistance and truncated thymidine kinase) flanked by ITR sequences was amplified by PCR from PB-MVI Puro-TK plasmid (Transposagen) using primers K1217 and K1218, and ligated with the linearized plasmid (see above) at a TTAA region 78 bp upstream of the insertion using Gibson assembly Master Mix (NEB). NEB® 5-alpha Competent E. coli cells were transformed with 2 ul of the assembly reaction product. Plasmid DNA from 10 colonies were isolated and sequenced using primer K1217 to confirm correct assembly.

Genome editing using sgRNA and donor plasmids: For in vitro replication of InDels found in kidney disease patients, cultured human podocytes derived from a single cell were transfected by electroporation (Biorad Gene Pulser Xcell™ Electroporation System, 0.2 cm cuvette, square wave mode, 150 V and 10 millisecond pulse) with the CRISPR/Cas9 vector containing the specific sgRNA, and a donor plasmid containing the donor sequence and the antibiotic selection cassette. Following removal of non-transfected cells by incubation with 1 μg/ml Puromycin Dihydrochloride (Gibco) for 15 days, 10 μg of Excision-only piggyBac transposase expression vector (Transposagene) was transfected for scarless removal of the antibiotic selection cassette. Four days after transfection, cells were incubated with 2.5 μM ganciclovir (Sigma) to remove cells with residual truncated thymidine kinase activity. Single cells were picked, clones established, genomic DNA extracted using QIAamp DNA Mini Kit (QIAgen) and the target region PCR amplified using Platinum HiFi DNA polymerase (Invitrogen) and primers K1189 and K1188. PCR products were gel purified using QIAquick Gel Extraction Kit (QIAgen), cloned into pCR2.1 vector using TA Cloning™ kit (Invitrogen) and the insert sequenced using the M13 Forward sequencing primer. Sequences were aligned with native podocyte genomic sequence and the donor template sequence by BLAST.

### CRISPR A

Overall methods were identical to those for CRISPR B, with the exception of primers and oligonucleotides used, and the following site-specific details: An 8 bp insertion (TGGATGGA) was introduced at Chr 8-122,304,094-122,304,095), and the sgRNA designed to recognize a specific site 73 bp upstream of the insertion site. While generating the donor plasmid, the patient specific genomic DNA (patient SF3) was cloned into the pBlueScript II KS+ vector between the Spel and BamHI sites. During Gibson assembly, the antibiotic resistance cassette was ligated with the linearized plasmid at a TTAA region 51 bp upstream of the insertion.

### STAT5, STAT6 and NFκB Pathway Studies in Animal Models

BALB/c and BALB/cJ mice (n=3 mice per group) were injected with normal saline or Cocktail D dose 3× and euthanized at 15-, 30- and 60-minute time points. Mice were perfused with protease inhibitors (Thermo Fisher Scientific, catalog number: A32953) and phosphatase inhibitors (Thermo Fisher Scientific, catalog number: A32957) via the left ventricle injection prior to euthanasia. For qualitative studies, total protein was extracted with RIPA buffer (Thermo Fisher Scientific, catalog number: 89900) from liver, heart and kidney in the presence of protease and phosphatase inhibitors to confirm activation of STAT5 (STAT5 and pSTAT5), STAT6 (STAT6 and pSTAT6) and NFκB (p65 and phospho-p65) pathways. For quantitative studies, nuclear and cytosolic fractions were separately extracted from these organs for each mouse separately using the Nuclear Extraction Kit (Nous Biological, Centennial CO, USA, cat #NBP-2-29447). Western blots for nuclear expressed protein Lamin B1 were conducted on both fractions to confirm predominant expression in the nuclear fraction. Western blot for GAPDH was conducted to confirm presence in both fractions. For relative quantitation by Western blot, pSTAT proteins were expressed as a ratio with Lamin B1 in nuclear fractions and the corresponding STAT protein in cytosolic fractions. Both ratio components were always scanned from the same non-saturated film image, and densitometry conducted using Bio-rad Image Lab 6.1 software with manual detection of close cropped bands, background subtraction, band identification, and adjusted total lane volume calculation. Antibodies against the following proteins were purchased: STAT5 (D2069, 1:500), p-STAT5 (D47E7, 1:1000), STAT6 (D3H4, 1:500), P-STAT6 (D8S9Y, 1:1000), NF-κB p-65 (D14E12, 1:1000), P—NF—κB P-p-65 (S536, 1:1000), GAPDH 14C10, 1:20,000), all from Cell Signaling Technology, Inc. Danvers MA, USA; Lamin B1 (ab16048, 1:5,000, Abcam Cambridge U.K.); Donkey anti Rabbit IgG HRP (1:20,000, Jackson Laboratories)

### In Vitro STAT6 Signaling Studies

Wild-type (precursor of CRISPR modified podocytes) and CRISPR-B podocytes were grown in RPMI 1640 media containing heat-inactivated 10% fetal bovine serum, 1% Insulin-Transferrin-Selenium (ITS-G, Thermo Fisher Scientific-catalog number 41400045) and 1% Penicillin-Steptomycin (Thermo Fisher Scientific, catalog number 15140122) at 33° C. Cells were subcultured and 50,000 cells/dish were seeded on 10 cm culture dishes at 37° C. for 3 days. Next, culture media were exchanged with RPMI 1640 containing heat-inactivated 0.2% FBS and 1% Penicillin-Steptomycin. After 24 hr, cells were treated with Cocktail C or Common Cold Cocktail (X/100,000) for 10, 20 and 30 min. Proteins were isolated with RIPA buffer (Thermo Fisher Scientific, catalog number: 89900) containing protease inhibitor (Thermo Fisher Scientific, catalog number: A32953) and phosphatase inhibitor (Thermo Fisher Scientific, catalog number: A32957) (10 ml of RIPA buffer contained 1 tablet each of protease and phosphatase inhibitor). Protein concentration was assessed using the Bradford protein assay.

Human Plasma from COVID-19 and Control Patients for IL-4Rα Assay

Human plasma 100 μL aliquots were obtained from the following sources (a) De-identified IRB approved hospitalized COVID patient samples from the Rush University COVID-19 Registry and Biorepository. (b) De-identified IRB approved hospitalized COVID patient samples from the Rush University COVID-19 Registry and Biorepository, selected for presence of proteinuria. (c) De-identified plasma samples that were age, sex and race matched to group a, purchased from Zenbio (Durham NC, USA).

### Statistical Analysis

Values in all graphs are mean+s. e. m. For difference in proteinuria, albuminuria or gene expression involving 2 groups, we used the unpaired Student's t test in Microsoft Excel 2013. Unless specifically indicated, all significance is two-tail.

Further reference is made to the following experimental examples.

### EXAMPLES

The following examples are provided for the purpose of illustrating various embodiments and are not meant to limit the present disclosure in any fashion. The present examples, along with the methods described herein are presently representative of preferred embodiments, are provided only as examples, and are not intended as limitations on the scope of the claimed subject matter. Changes therein and other uses which are encompassed within the spirit of the disclosure as defined by the scope of the claims will occur to those skilled in the art.

### Example 1

### Developing Novel COVID-19 Cytokine Storm Cocktails

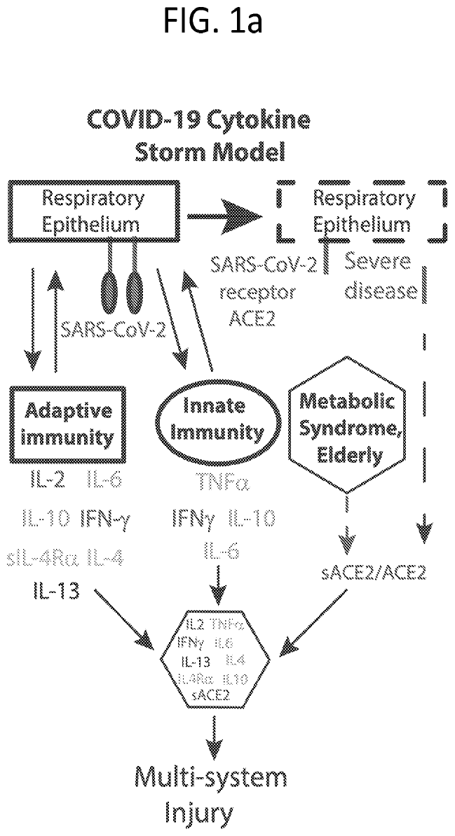

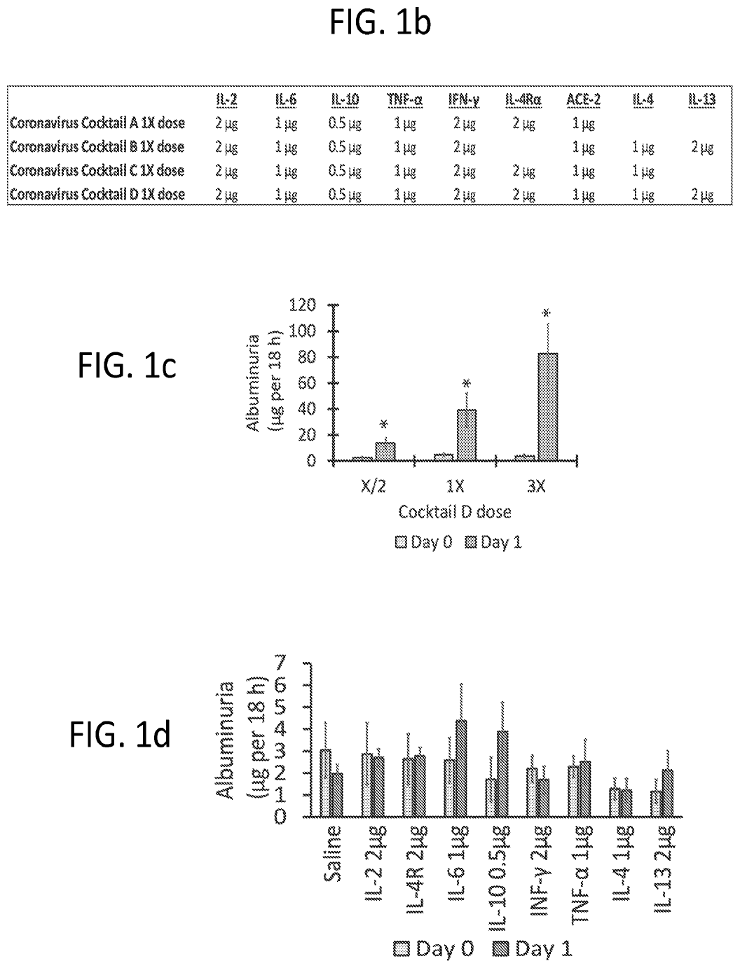

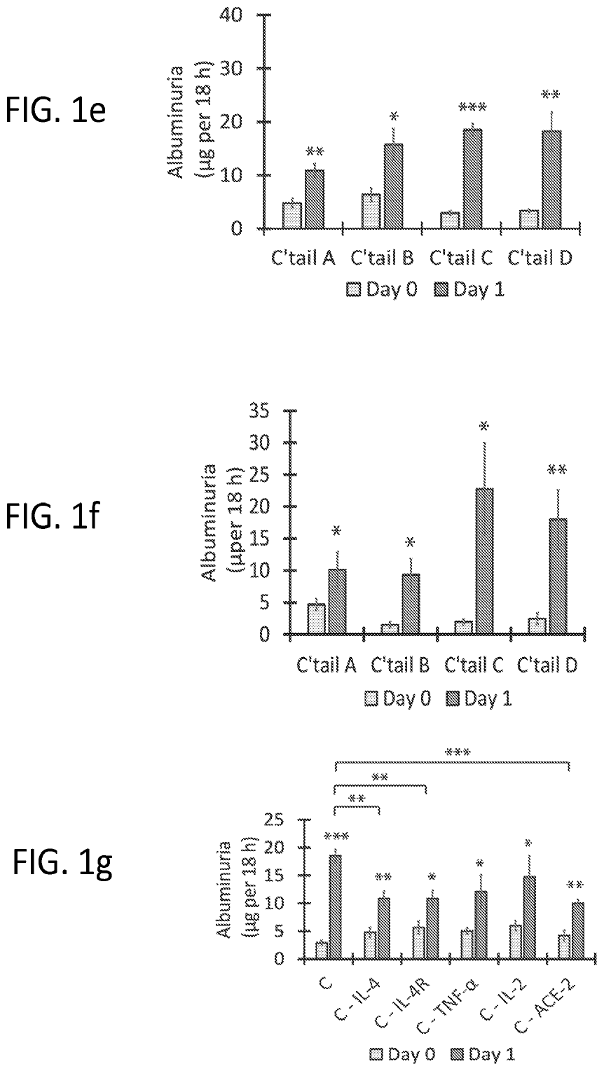

FIG. 1a-g depicts the development and characterization of COVID-19 cytokine storm models. (FIG. 1a) Schematic representation of COVID-19 induced cytokine storm in the context of human disease. (FIG. 1b) Composition of dose X of the COVID cocktails A to D. (FIG. 1c) Albuminuria after injecting different doses of Cocktail D into BALB/cJ mice (n=4 mice per group). X/2 is the threshold nephritogenic dose in BALB/cJ mice. (FIG. 1d) Albuminuria after injecting dose X of individual COVID cocktail components in BALB/cJ mice (n=4 mice per group). (FIG. 1e) Albuminuria after injecting COVID cocktails A to D dose X/2 in BALB/c mice (n=6 mice per group). (FIG. 1f) Albuminuria after injecting COVID cocktails A to D dose X/2 in BALB/cJ mice (n=6 mice per group). (FIG. 1g) Albuminuria after injecting BALB/c mice with intact Cocktail C dose X/2 or Cocktail C dose X/2 lacking individual components that target podocytes (n=6 mice per group). P<0.05; **P<0.01; ***P<0.001. All significant values are two-tail.

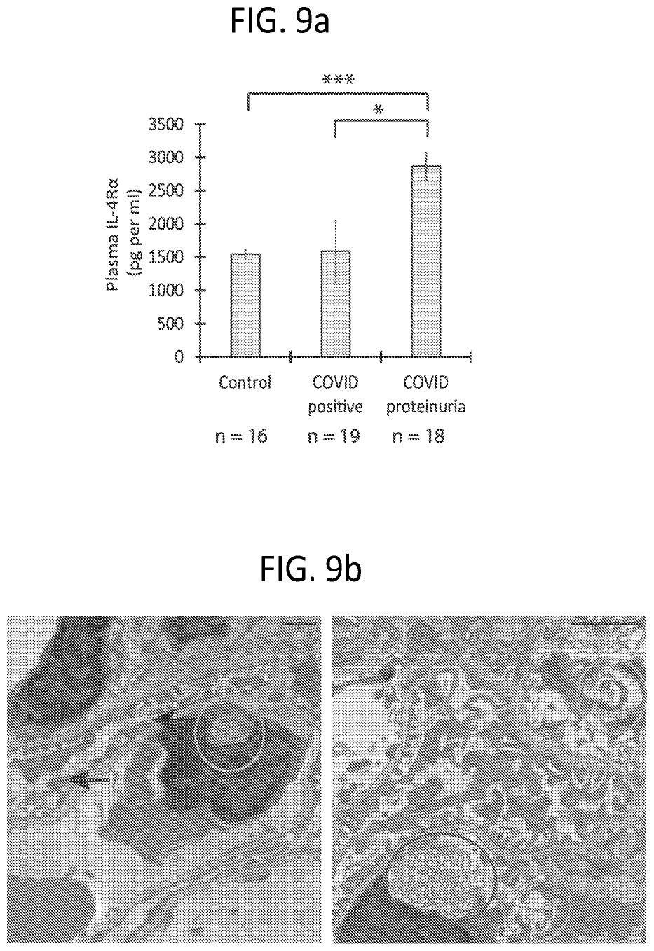

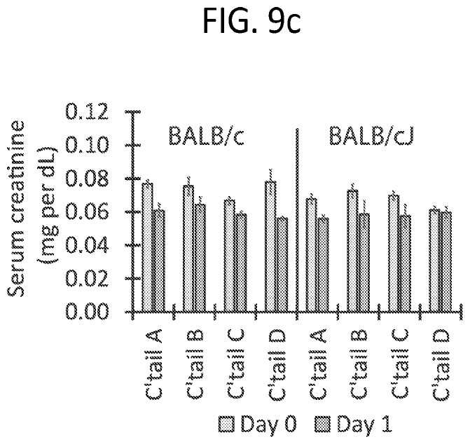

FIG. 9a-c: (FIG. 9a) Plasma IL-4Rα levels assessed by ELISA in general COVID-19 patients, age, sex and race matched healthy controls, and COVID-19 patients with proteinuria. Number of patient samples assayed is shown below. (FIG. 9b) Electron microscopy images of BALB/cJ mouse glomeruli on Day 1 after injection of Cocktail D dose X/2. Areas of focal foot process effacement (black arrows), endothelial vacuolation (green circles), and endothelial hypertrophy (blue circles) were noted. (FIG. 9c) Serum creatinine, assayed by Mass Spectrometry, is not increased in the COVID cytokine cocktail dose X/2 models (BALB/c and BALB/cJ mice; n=6 mice per group). Scale bars 0.5 μm. *P<0.05; **P<0.01.

COVID cocktails A to D were developed in a stepwise manner to model the hospitalized COVID-19 patients in intensive care (FIG. 1b). The first 5 cytokines (FIG. 1b) are common to all cocktails. Circulating IL-4Rα levels are also increased in COVID patients with proteinuria (FIG. 9a) ACE2, the COVID-19 receptor, was included in COVID-19 cocktails since plasma sACE2 levels are significantly higher in sick COVID-19 patients in Intensive Care, and in elderly and metabolic syndrome patients who are predisposed to severe COVID-19 disease. High plasma IL-13 and IL-4 levels in sick COVID-19 patients points towards acute activation of the allergy pathway in this disease. Removing sIL-4Rα from Cocktail A and adding IL-4 and IL-13 made cocktail B, whereas adding IL-4 to Cocktail A gave Cocktail C. Adding IL-13 to Cocktail C gave Cocktail D.

A dose-response study showed X/2 to be the threshold nephritogenic dose in BALB/cJ mice (FIG. 1c) that also induced histological changes (FIG. 9b). Individually, none of these cytokines used in the same dose as in combination X caused albuminuria (FIG. 1d). Lower baseline albuminuria in BALB/cJ Vs. BALB/c mice has been reported. See Macé et al. 2020.

### Example 2

### Systemic Manifestations of Synergistic Multi-Cytokine Injury Induced by COVID-19 Cocktails

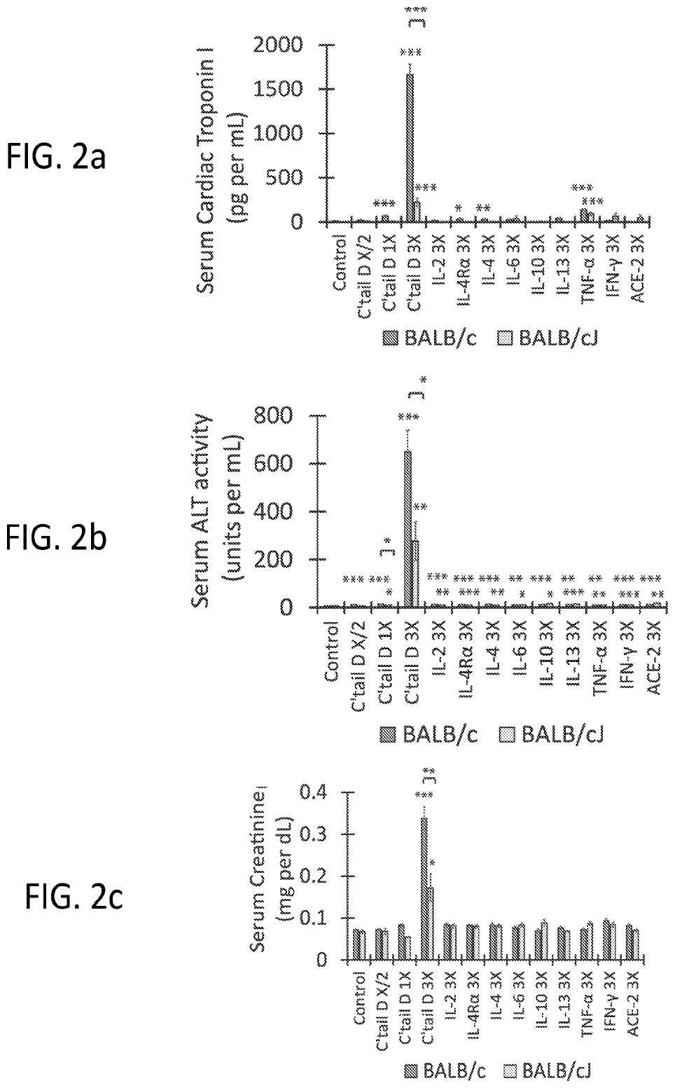

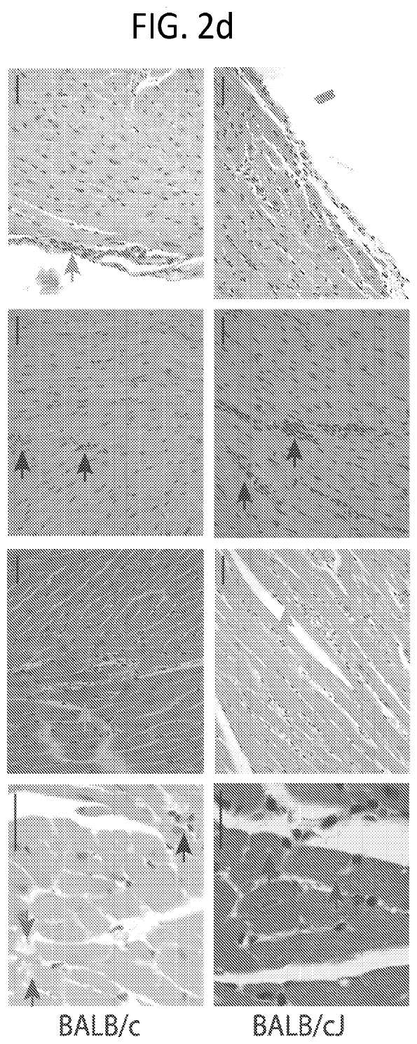

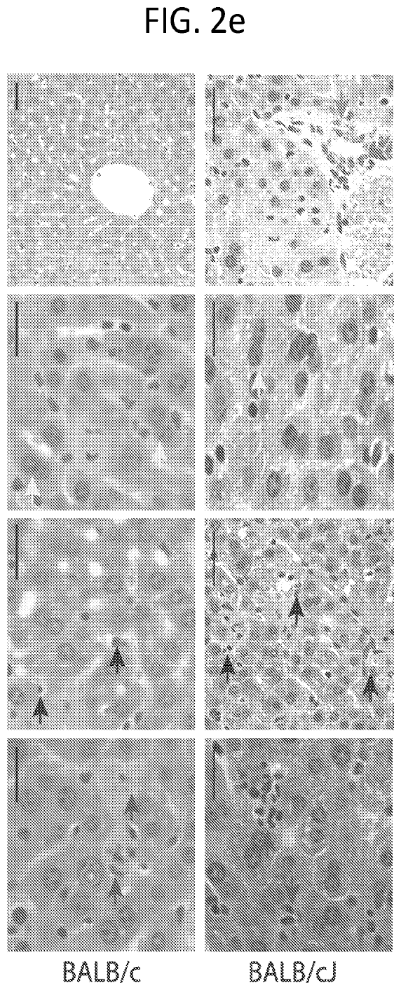

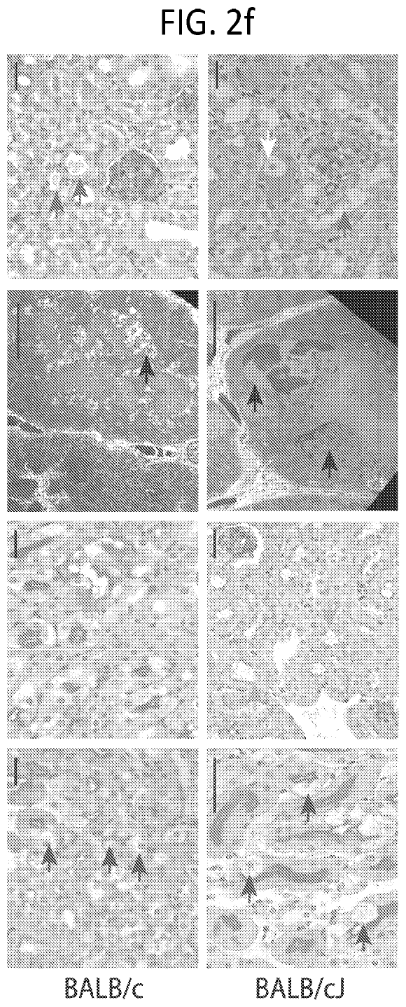

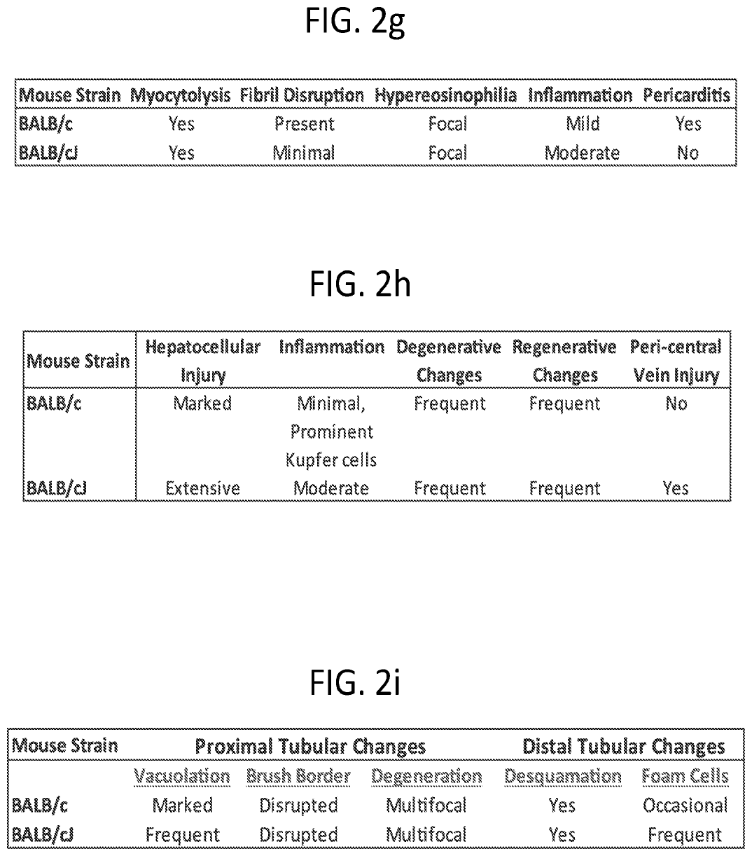

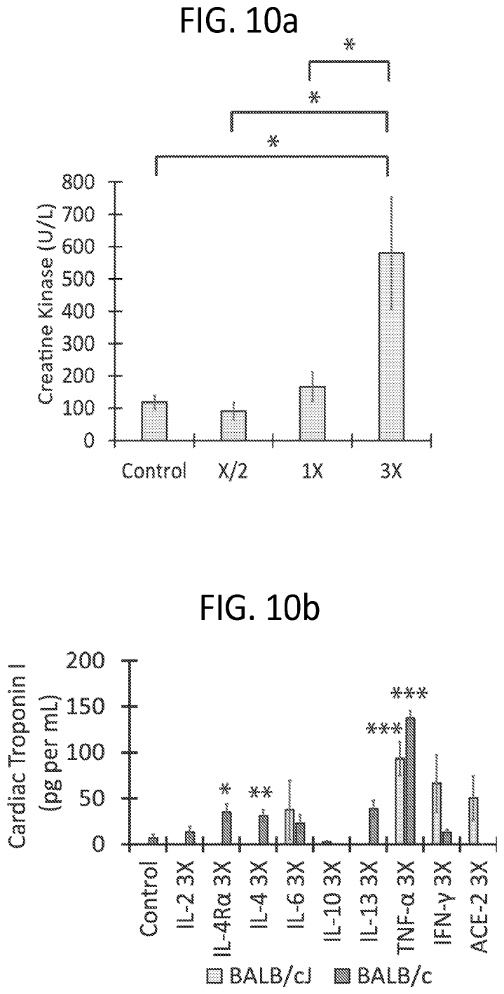

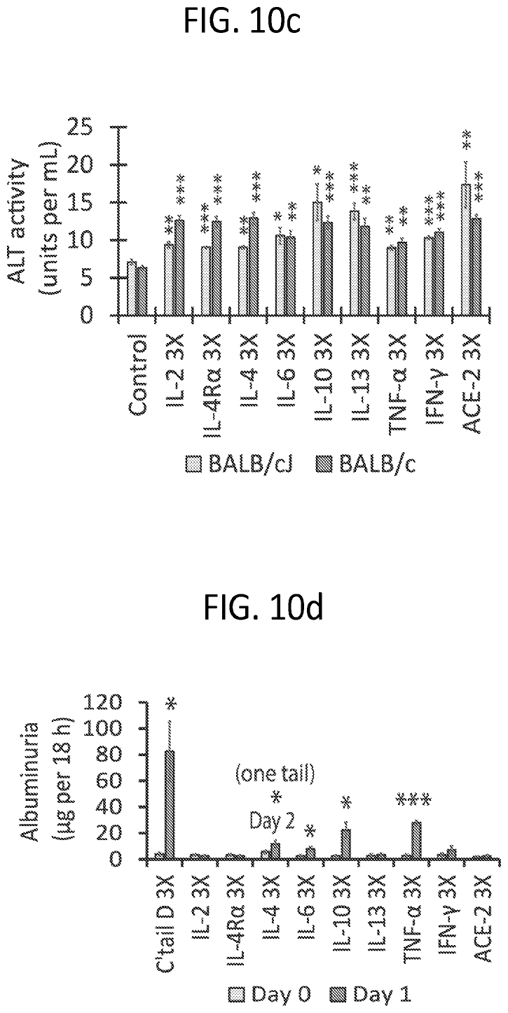

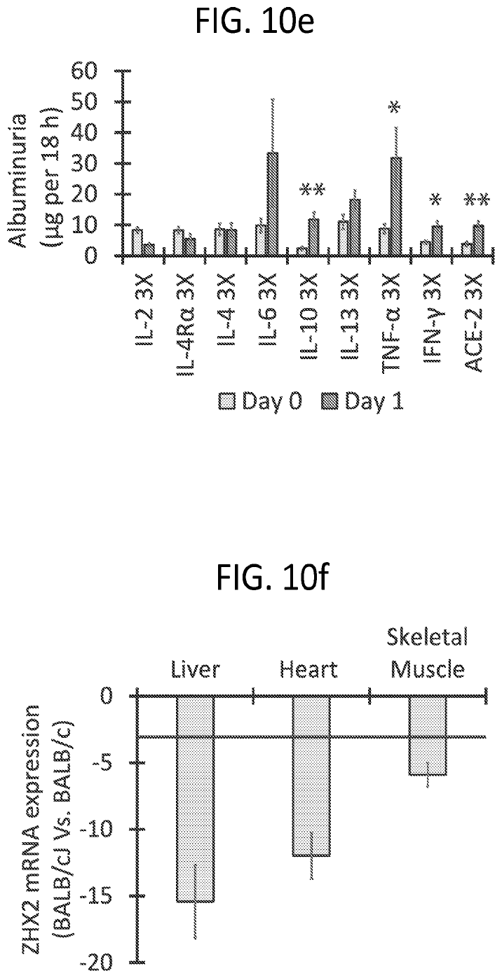

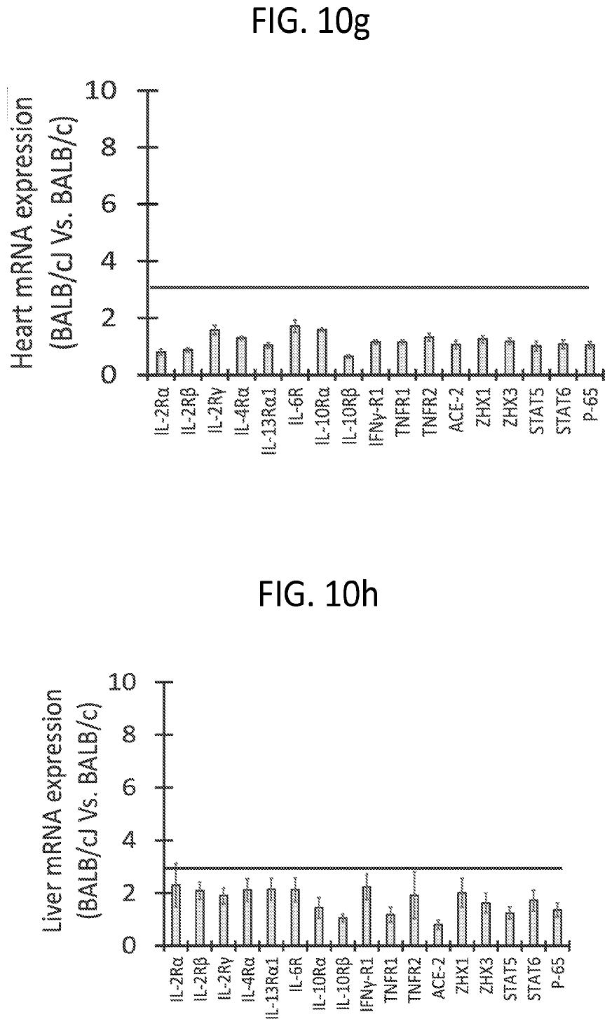

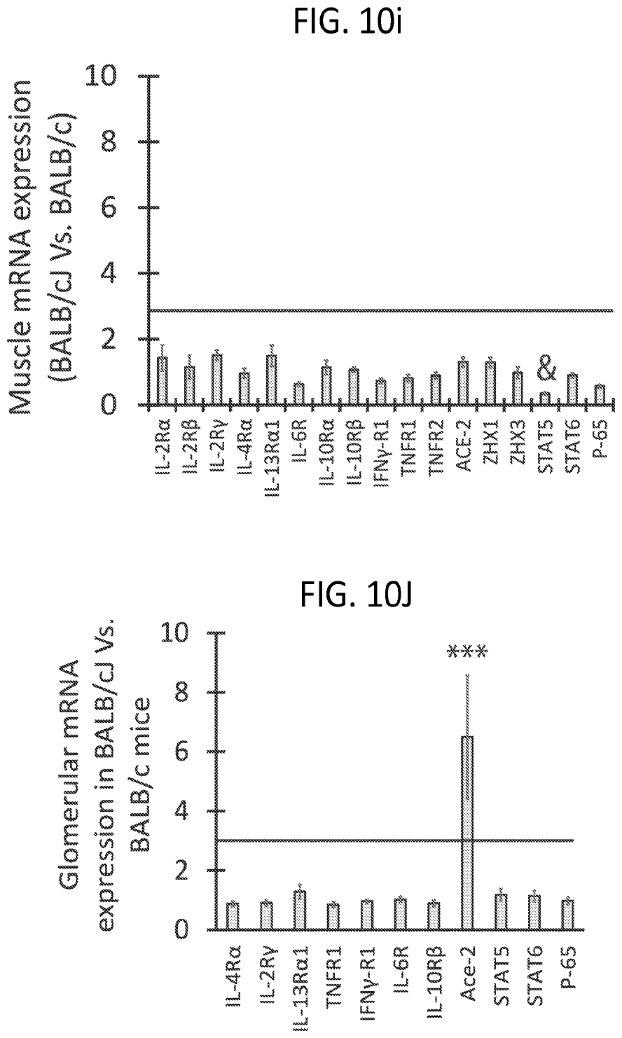

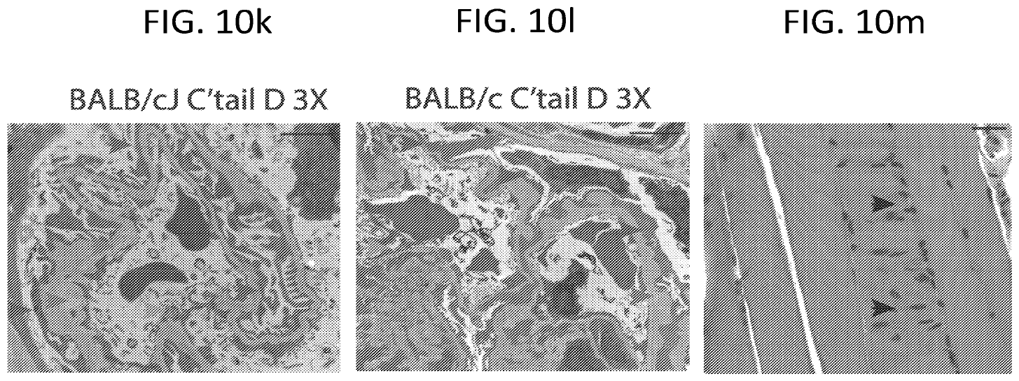

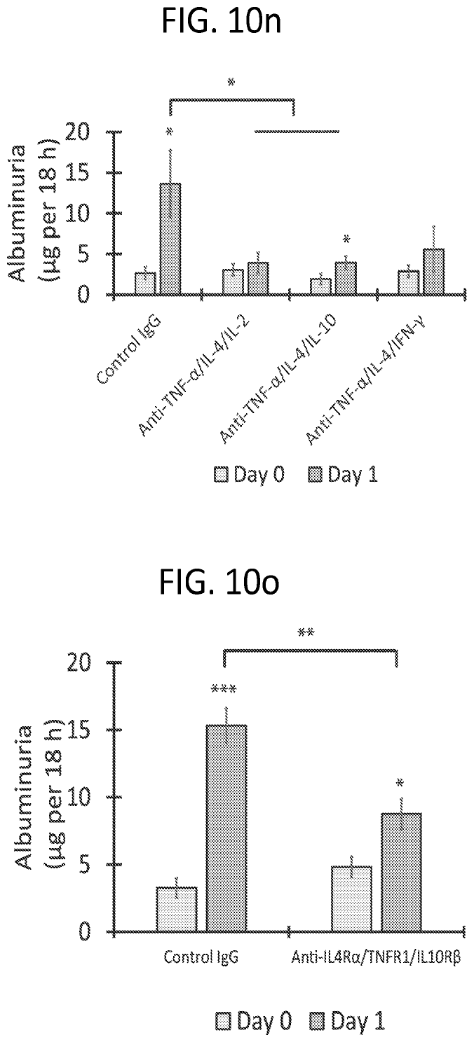

Injection of higher doses (3×) of Cocktail D induced albuminuria as well as causing elevation of serum cardiac Troponin I Type 3 (cTPI3; myocardial injury, FIG. 2a), serum alanine aminotransferase (ALT, acute liver injury, FIG. 2b), serum creatinine (Acute Kidney Injury, AKI; FIG. 2c), and plasma creatine kinase (CK, skeletal muscle injury; FIG. 10a).

FIG. 2a-h depicts reduced systemic injury induced by high dose of Cocktail D (3×) in ZHX2 hypomorph BALB/cJ compared to ZHX2 BALB/c mice. Dose 3× also has higher injury compared to lower doses or individual components at dose 3×. (FIG. 2a) Acute myocardial injury assessed by cardiac Troponin I levels (cTPI3) levels (n=8 mice per group). (FIG. 2b) Acute liver injury assessed by alanine aminotransferase (ALT) activity levels (n=8 mice per group). (FIG. 2c) Acute kidney injury assessed by serum creatinine measured using mass spectrometry (n=8 mice per group). (FIG. 2d) Histological characterization of acute cardiac injury (n=3 mice per group) using H&E-stained sections from Cocktail D dose 3× injected mice. Myocytolysis (red arrows), inflammation (black arrows), fibril disruption (blue arrows), hypereosinophilia (green arrows) and pericarditis (orange arrow) were noted. (FIG. 2e) Histological characterization of acute liver injury (n=3 mice per group) using H&E-stained sections from Cocktail D dose 3× injected mice. Hepatocellular injury (red arrows), inflammation (black arrows), prominent Kupfer cells (green arrows), regenerative changes (yellow arrows) and peri-central vein injury (blue arrow) were noted. (FIG. 2f) Histological assessment of acute kidney injury (n=3 mice per group) using PAS-dose 3× injected mice. Lower three rows show proximal tubules, top row shows distal tubules. In proximal tubules, vacuolation (red arrows), brush border disruption (green arrows) and tubular degeneration (black arrows) were noted. In distal tubules, evidence of desquamation (blue arrows) was present. Foam cells were also noted (white arrows). Electron microscopy scale bars BALB/c, 2.66 μm; BALB/cJ, 2 μm. (FIG. 2g) Tables showing morphometric analysis and comparison of histological changes in the heart in BALB/c and BALB/cJ mice. (FIG. 2h) Tables showing morphometric analysis and comparison of histological changes in the liver in BALB/c and BALB/cJ mice. (FIG. 2i) Tables showing morphometric analysis and comparison of histological changes in the kidney in BALB/c and BALB/cJ mice. Light microscopy scale bars 20 μm. * P<0.05; **P<0.01; ***P<0.001, all values based on two-tail analysis.

FIG. 10a-n shows the characterization of ZHX2 hypomorph state in BALB/cJ mice: (FIG. 10a) Plasma creatine kinase, a marker of skeletal muscle injury, in BALB/cJ mice (n=4 mice per group) 24 hours after injection of Cocktail D at different doses. (FIG. 10b) Serum Cardiac Troponin I level data derived from FIG. 2a, plotted again for higher resolution of lesser increase in levels among some single cytokine injected groups. (FIG. 10c) Serum ALT level data derived from FIG. 2b, plotted again for higher resolution of lesser increase in levels among some single cytokine injected groups. (FIG. 10d) 18-hour albuminuria in BALB/cJ mice injected with Cocktail D 3× or single cytokines dose 3×, corresponding to FIGS. 2a-c. (FIG. 10e) 18-hour albuminuria in BALB/c mice injected with single cytokine dose 3×, corresponding to FIGS. 2a-c. Given their high mortality after Cocktail D 3×, metabolic cage housing for timed urine collection is not feasible in BALB/c mice. (FIG. 10f) Fold-difference in heart and skeletal muscle ZHX2 mRNA expression in BALB/cJ compared to BALB/c mice assessed by real time PCR (n=6 templates per group). Lower levels of ZHX2 mRNA expression in the liver were previously published and serve as a positive control for this phenomenon. Three-fold difference was taken as significant. (FIG. 10g) Real time PCR comparison of expression of cytokine receptors, Ace2, Zhx1, Zhx3, and signaling pathway proteins STAT5, STAT6 and P-65 (NFκB) between BALB/cJ and BALB/c mouse heart (n=6 templates per group). Three-fold difference was taken as significant. (FIG. 10h) Real time PCR comparison of expression of cytokine receptors, Ace2, Zhx1, Zhx3, and signaling pathway proteins STAT5, STAT6 and P-65 (NFκB) between BALB/cJ and BALB/c mouse liver (n=6 templates per group). Three-fold difference was taken as significant. (FIG. 10i) Real time PCR comparison of expression of cytokine receptors, Ace2, Zhx1, Zhx3, and signaling pathway proteins STAT5, STAT6 and P-65 (NFκB) between BALB/cJ and BALB/c mouse skeletal muscle (n=6 templates per group). Three-fold difference was taken as significant. & 3.06+0.40 fold higher expression of STAT5 in BALB/c mice. (FIG. 10j) Real time PCR comparison of expression of cytokine receptors, Ace2, and signaling pathway proteins STAT5, STAT6 and P-65 (NFκB) between BALB/cJ and BALB/c mouse glomeruli (n=6 templates per group). Three-fold difference was taken as significant. Other IL-2R chains are not expressed in mouse glomeruli, and Zhx1 and Zhx3 are previously published. (FIG. 10k) Electron microscopy of BALB/cJ mouse kidney glomeruli 24 hours after injection Cocktail D dose 3×. Multifocal foot processes effacement (red arrows), endothelial hypertrophy (green arrows) and glomerular basement membrane (GBM) remodeling (blue arrows) were present. (FIG. 10l) Electron microscopy of BALB/c mouse kidney glomeruli 24 hours after injection Cocktail D dose 3×. Extensive foot processes effacement (red arrows), endothelial hypertrophy (green arrows) and glomerular basement membrane (GBM) remodeling (blue arrows) were present. (FIG. 10m) Hematoxylin and Eosin-stained skeletal muscle from BALB/cJ mice 24 hours after injection of Cocktail D dose 3×. Focal inflammation (black arrows) was noted in some sections. (FIG. 10n) Albuminuria after induction of the Cocktail D model in BALB/cJ mice (n=6 mice per group; dose X/2), followed by Control IgG or combinations of depleting antibodies one hour after model induction. (o) Albuminuria after induction of Cocktail C in BALB/c mice (n=6 mice per group; dose X/2), followed by receptor blockage using antibodies against IL-4Rα, TNFR1 and IL-10Rβ, or control IgG. Scale bars (k) 0.5 μm, (1) 0.5 μm, (m) 20 μm. *P<0.05; **P<0.01; ***P<0.001.

cTPI3, ALT and albuminuria also increased at 3× dose for some individual cytokines, albeit at a significantly lower level than the cocktail (FIG. 2a, b; FIG. 10b-e). Timed urine collection in metabolic cages for albuminuria assessment was not conducted for Cocktail D 3× dose injected BALB/c mice in view of high mortality (see below). Cocktail D 3× dose induced significantly more severe cardiac, liver and acute kidney injury in BALB/c compared to BALB/cJ mice, suggesting that the latter are protected by the ZHX2 hypomorph state. While liver, kidney and glomerular ZHX2 hypomorph state in BALB/cJ mice is previously described, (Macé et al. 2020; Perincheri et al., 2005; Perincheri et al., 2008) the inventors found similar changes in the heart and skeletal muscle (FIG. 10f). mRNA expresion of cytokine receptors, ACE2, other ZHX proteins and select signaling pathway proteins in heart, liver, skeletal muscle and glomeruli was similar between BALB/cJ and BALB/c mice (FIG. 10g,h,I,j; exceptions, higher Ace2 in BALB/cJ glomeruli, higher STAT5 in BALB/c skeletal muscle). Cardiac histology (FIG. 2d) revealed myocytolysis, focal fibrillar disruption and hypereosinophilia, inflammation (myocarditis) and pericarditis. Liver histology (FIG. 2e) showed substantial hepatocellular injury, prominent Kupfer cells, frequent degenerative and regenerative changes, and mild inflammation. Histological evaluation of the kidney tubulo-interstitial compartment (FIG. 2f) revealed evidence of proximal tubular injury in the form of frequent vacuolation, luminal widening, brush border disruption and desquamation of tubular epithelial cells. Desquamation of epithelial cells, foam cells and vacuolation were also noted in distal tubules. Morphometric differences in these organs 24 hours after injection of Cocktail D 3× were noted between BALB/c and BALB/cJ mice (FIG. 2g, h, i). However, no evidence of severe or extensive inflammation was seen.

### Example 3

Effect of ZHX2 Expression on Mortality after Induction of the Severe Cytokine Storm Model (Cocktail D Dose 3×).

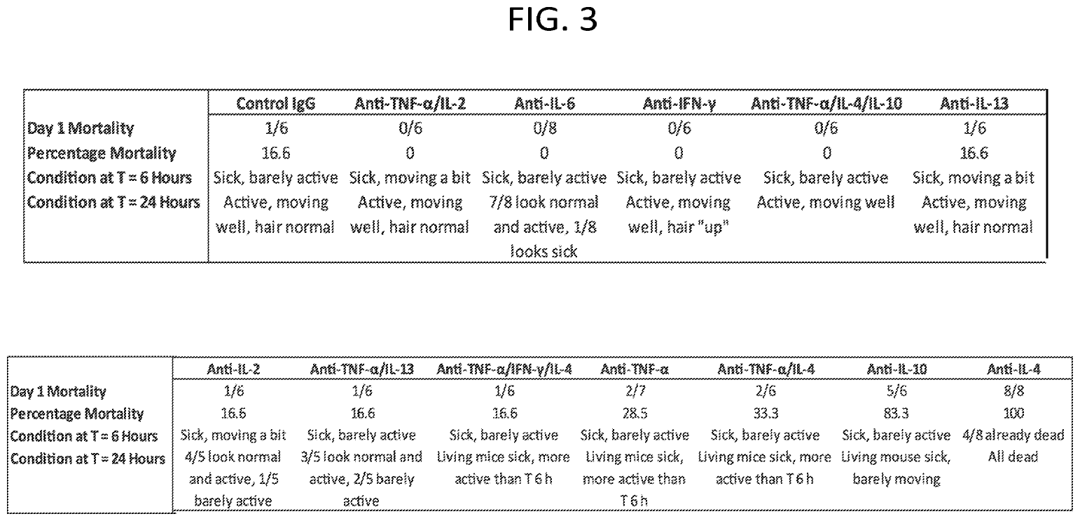

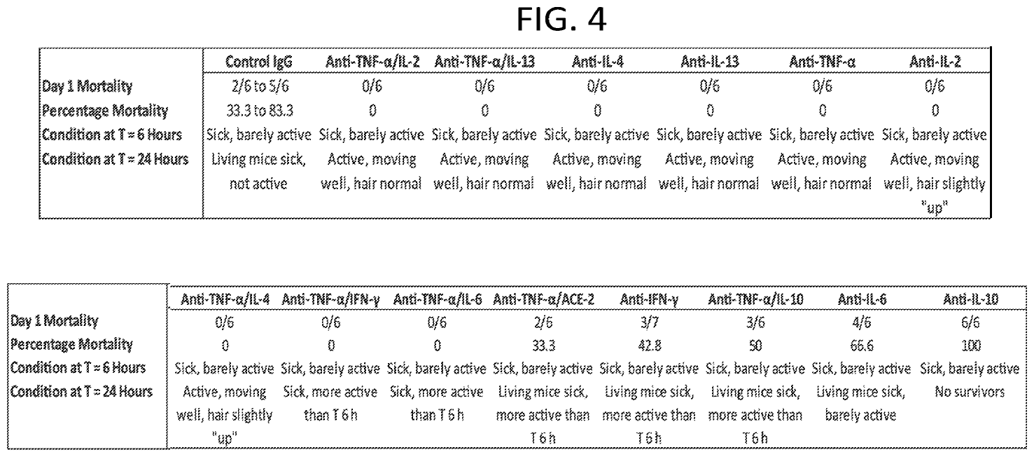

FIG. 3 and FIG. 4 show mortality in ZHX2 hypomorph BALB/cJ mice (FIG. 3) and ZHX2 BALB/c (FIG. 4) without or after cytokine depletion. Number of mice injected per group are shown in the denominator. The Control IgG group in each figure represent mortality in each strain without any therapeutic cytokine depletion. Under comparable housing conditions that include metabolic cages, mortality in this group is much higher in BALB/c (5/6 mice or 83.3%; FIG. 4) compared to BALB/c mice (1/6 mouse or 16.6%; FIG. 3). The rest data in FIG. 4 was generated by housing mice in normal cages, which was associated with lower mortality of 2/6 or 33.3%. All depleting antibodies or control IgG were injected intravenously one hour after model induction.

### Example 4

### Genomic Basis of the ZHX2 Hypomorph State

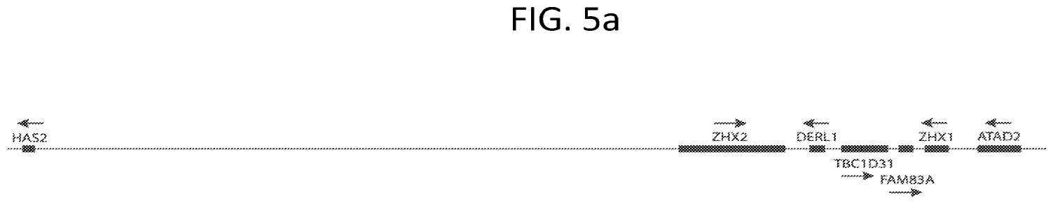

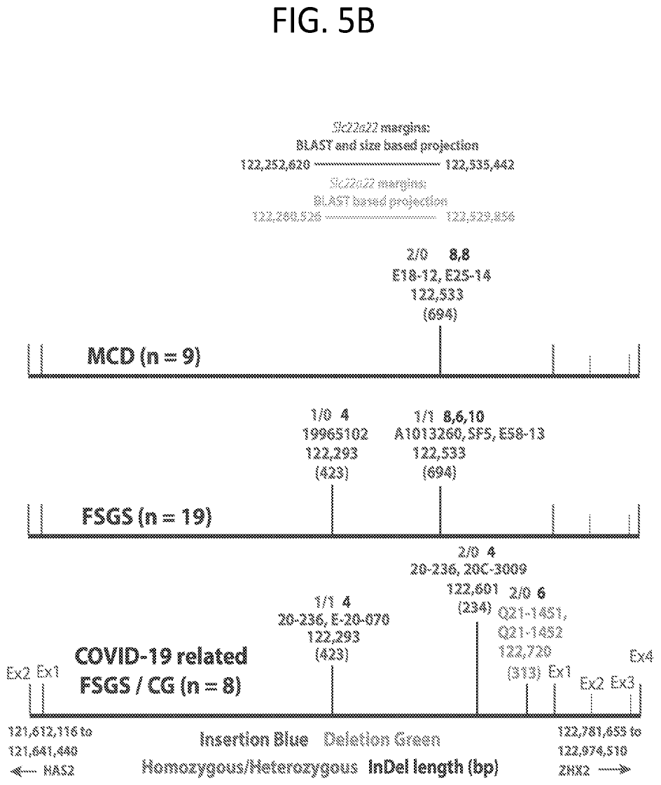

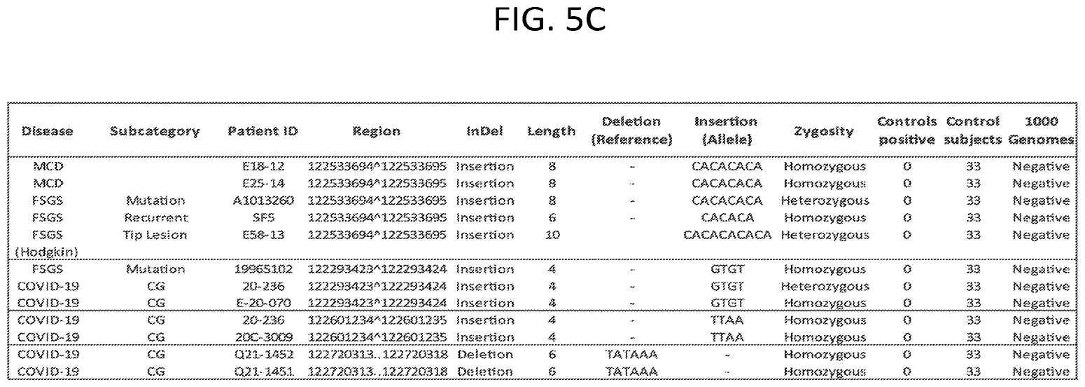

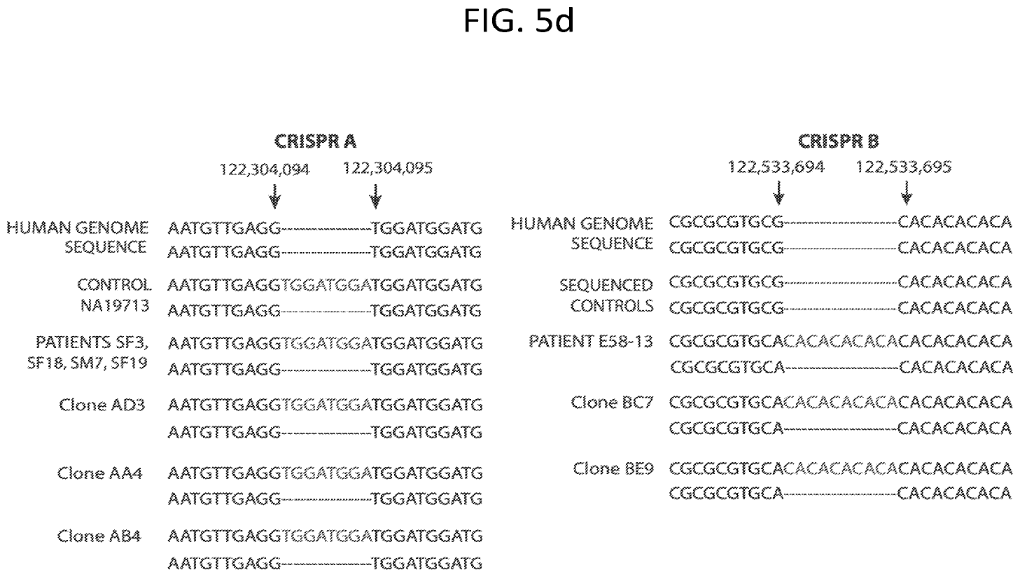

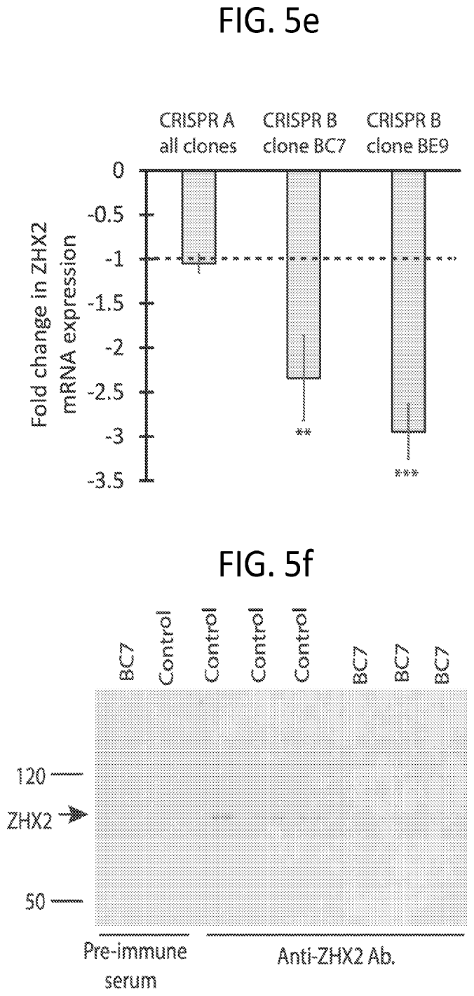

Since large scale whole exome sequencing studies did not identify any ZXH2 related disease-causing variants MCD, FSGS, COVID-19 related FSGS collapsing variant) and control subjects were sequenced from the beginning of HAS2 (the immediate upstream gene) (FIG. 5a) to the end of ZHX2. The 1000 genomes database was used as an additional control.

FIG. 5a-f: Genomic basis of the ZHX2 hypomorph state. Insertions and deletions (InDels) in non-coding DNA affect ZHX2 expression in patients with glomerular disease. (FIG. 5a) Schematic representation of ZHX2 and neighboring genes on chromosome 8. (FIG. 5b) Mapping of shared insertions and deletions among patients with MCD (n=9 patients), FSGS (n=19 patients), and COVID-19 related FSGS collapsing variant (n=8 patients). The most common shared insertion at 122,533,694 could be mapped at or close to the theoretical beginning of the rodent expressed gene S1c22a22 that is defunct in humans. Another shared insertion at 122,293,423 was located close to the theoretical end of S1c22a22. None of these InDels were noted in control subjects (n=33) or the 1000 Genomes project. Ex is Exon. (FIG. 5c) Tabular representation of shared insertions discussed above. (FIG. 5d) Schematic representation of CRISPR Cas9 assisted genome edited clones of a single cell derived cultured human podocyte cell line that contain an 8 bp insertion common between patients and a control subject (CRISPR A), or a 10 bp shared insertion at 122,533,694 that was absent in control subjects and the 1000 genomes project (CRISPR B). (FIG. 5e) Relative ZHX2 mRNA expression in above genome modified clones (CRISPR A, 3 clones, data pooled; study CRISPR B, 2 clones) compared to the parent single cell derived cultured human podocyte cell line (n=7 templates per clone). Dotted line represents expression in the parent cell line. (FIG. 5f) Western blot comparing ZHX2 expression in the Control single cell derived parent cell line and one of two mutant clones with insertion at 122,533,694. **P<0.01; ***P<0.001, all values based on two-tail analysis.

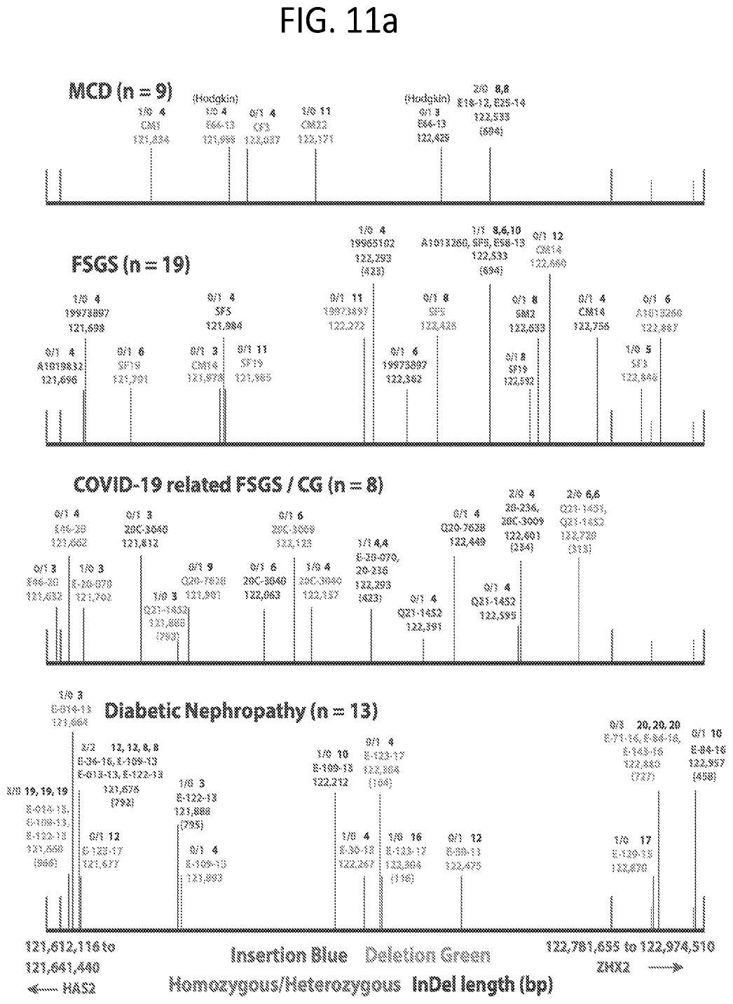

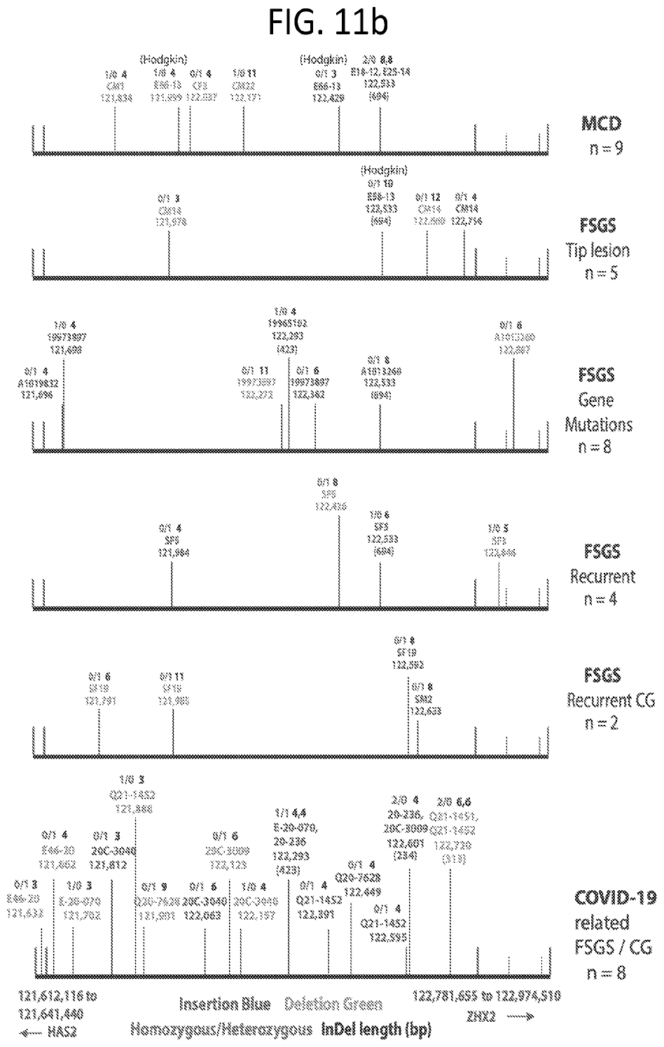

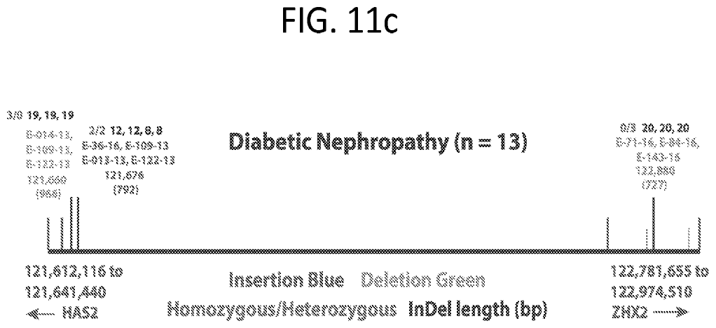

FIG. 11a-c: (FIG. 11a) Single and shared Insertions and Deletions (InDels) in the study population (FIG. 11b) InDels in FSGS patients expanded by disease sub-categories. (FIG. 11c) Shared InDels in diabetic nephropathy patients (n=13).

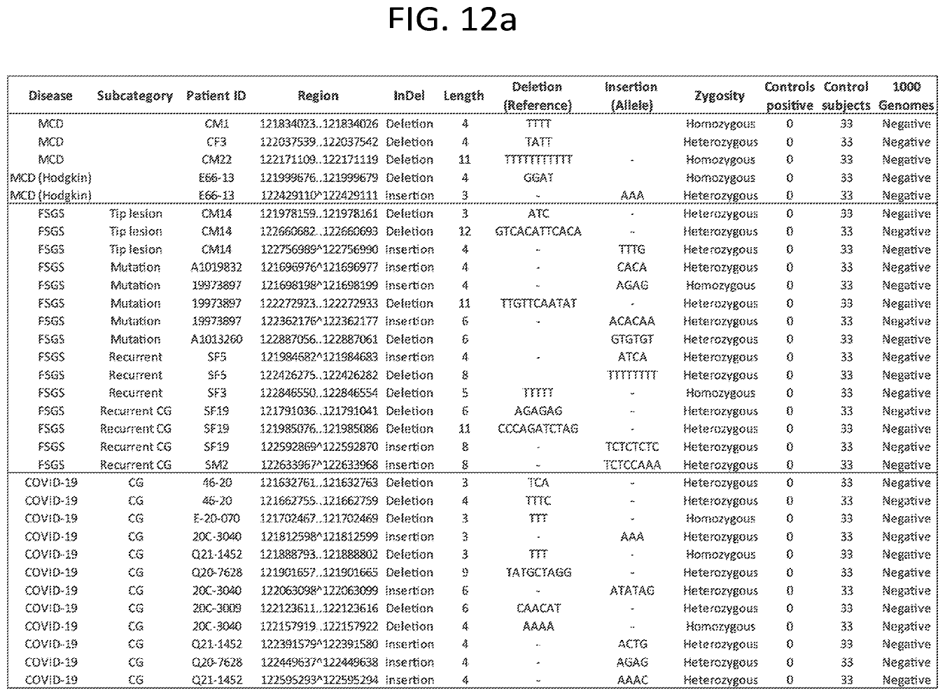

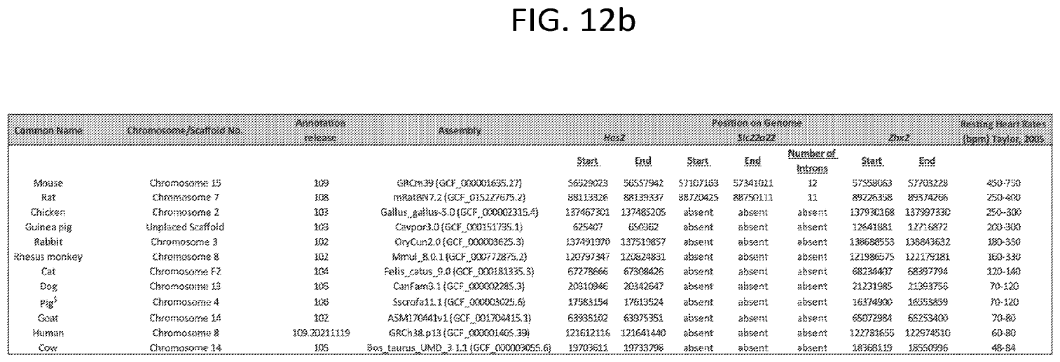

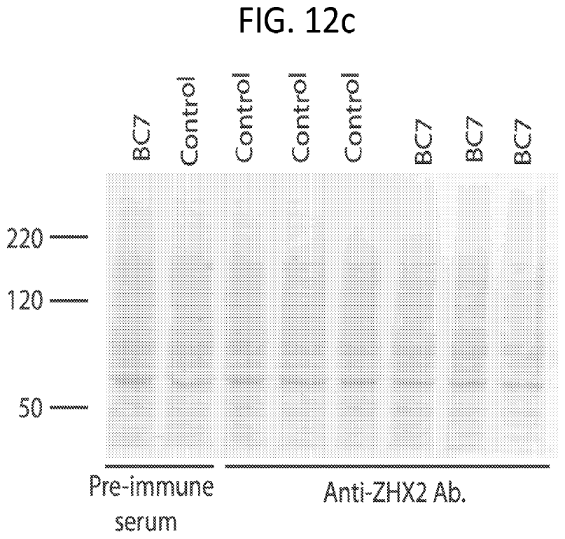

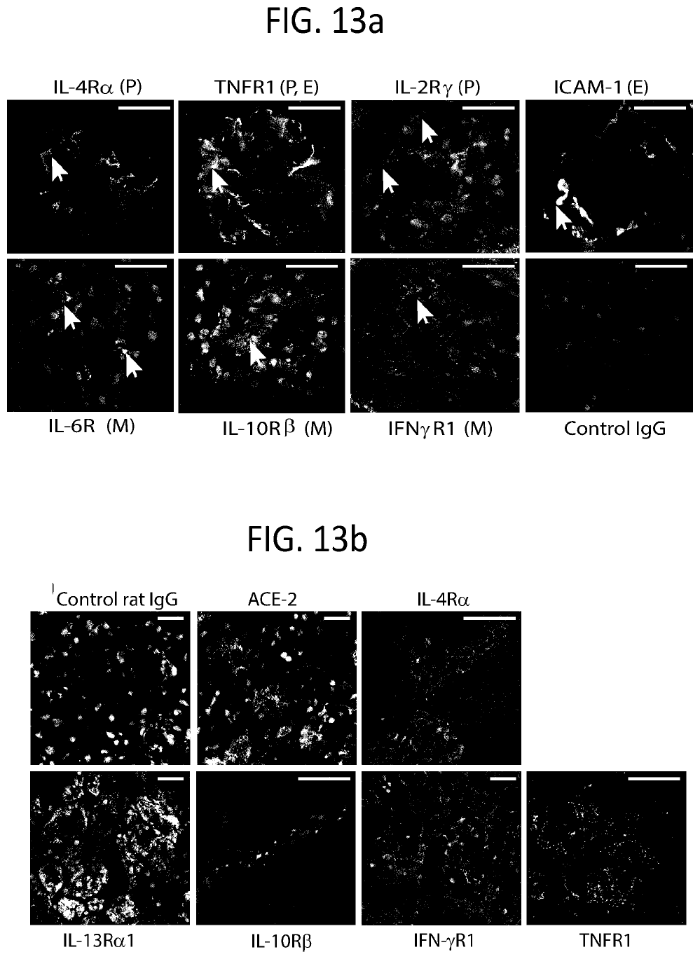

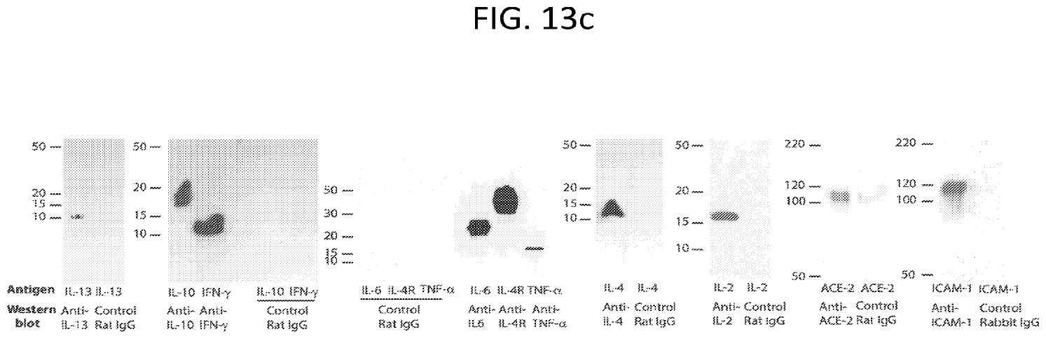

FIG. 12a-c: (FIG. 12a) List of single InDels in the study population. (FIG. 12b) The S1c22a22 gene between Has2 and ZHX2 in rodents, and its absence in larger animals and humans, arranged by size and heart rate. (FIG. 12c) Ponceau Red stained membrane from blot shown in FIG. 5f.

Multiple insertions and deletions (InDels), 3 bp or larger, noted exclusively in the patient population using CLC Genomics software were confirmed using IGV as a second screening method, and only InDels present by both methods were included. Six of 9 MCD patients, 10 of 19 FSGS patients, and all 8 COVID-19 CG patients had InDels. Three insertions and one deletion were shared by two or more patients. The insertion at 122,533,694 was present exclusively in patients with primary MCD, primary FSGS, or Hodgkin Lymphoma FSGS tip lesion.

Interspecies analysis of the genome showed the presence of the gene S1c22a22 between HAS and ZHX2 in mice and rats, but non-functional in higher species including humans. Fine mapping and analysis of remnants of the mouse S1c22a22 (a prostaglandin transporter) in the human genome showed the shared insertion at 122,533,694 as being present at or close to the origin of this gene site. None of the above shared insertions were noted in patients with diabetic nephropathy, the most common glomerular disease associated with chronic kidney disease in the western world (FIG. 11c).

To explore further the relative resistance of the ZHX2 hypomorph state to non-glomerular manifestations of COVID-19 cytokine storms and prior documentation of low podocyte ZHX2 expression in human MCD and FSGS, the insertion at Chromosome 8 122,533,694 was replicated in a single cell derived cultured human podocyte cell line using CRISPR-Cas9 technology (study CRISPR B, FIG. 5d). For comparison, another insertion noted in patients and control (control CRISPR A) was also replicated. ZHX2 mRNA expression was unchanged in all CRISPR A cell line clones (data pooled), and significant downregulation was noted in both clones generated for CRISPR B (FIG. 5e). Reduced expression of ZHX2 protein in a CRISPR B line compared to the parent cell line was noted on Western blot (FIG. 5f, FIG. 12c).

### Example 5

### Asynchronous Activation of Cell Signaling Pathways in ZHX2 Hypomorph Mice Compared to ZHX2^{+/+} Mice.

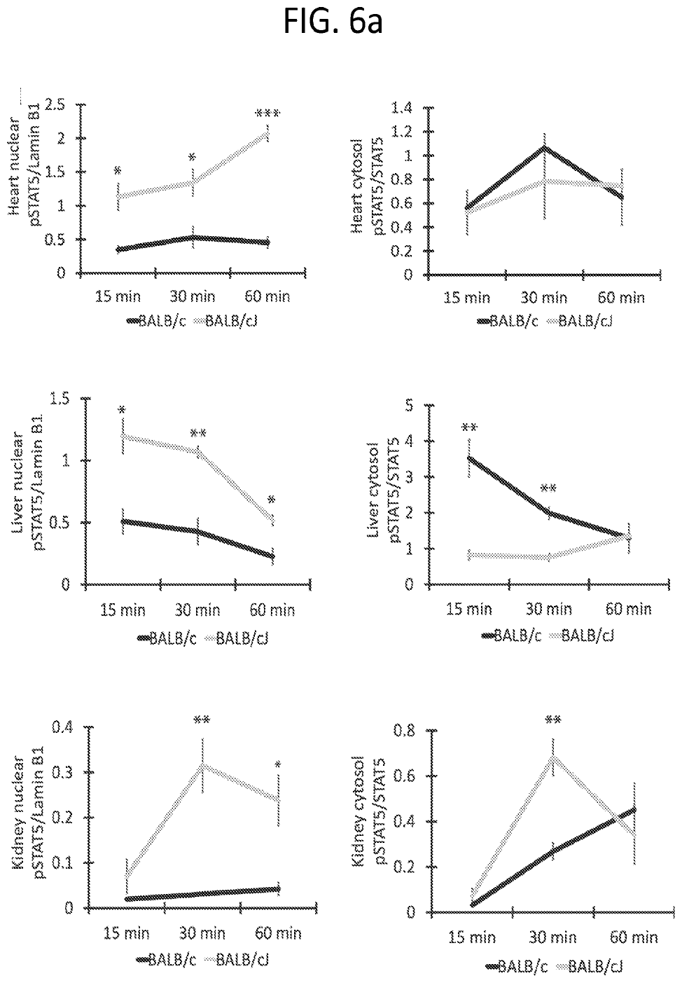

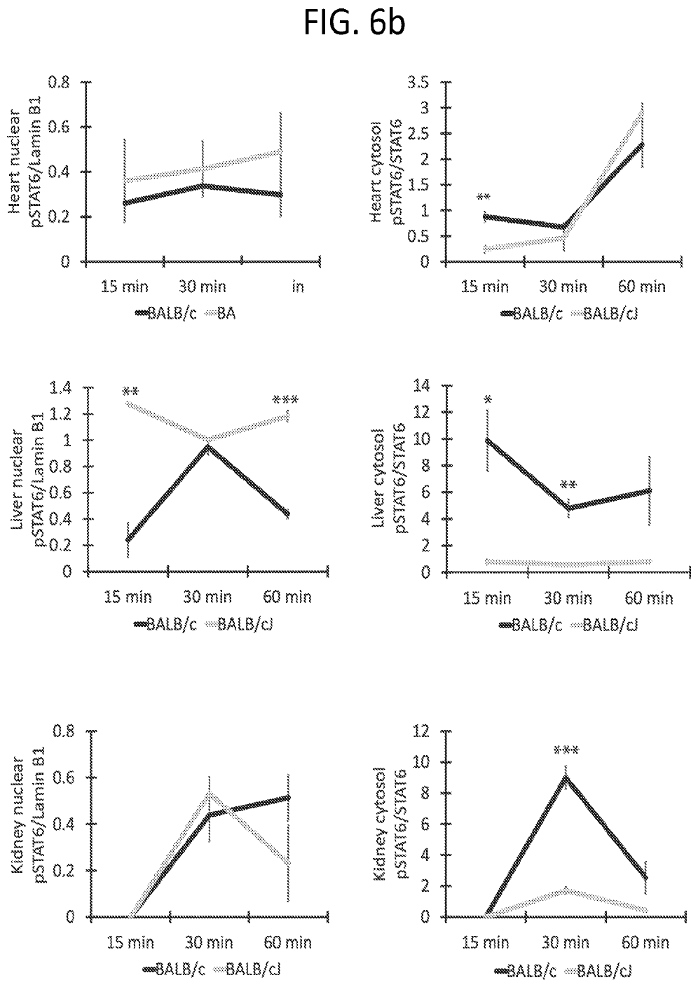

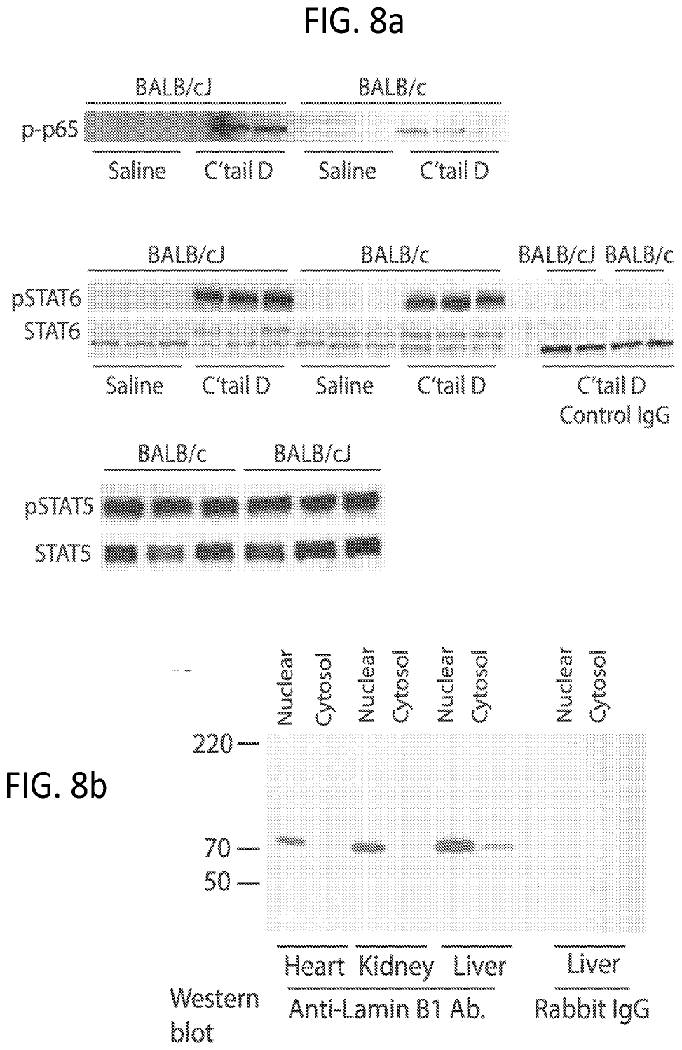

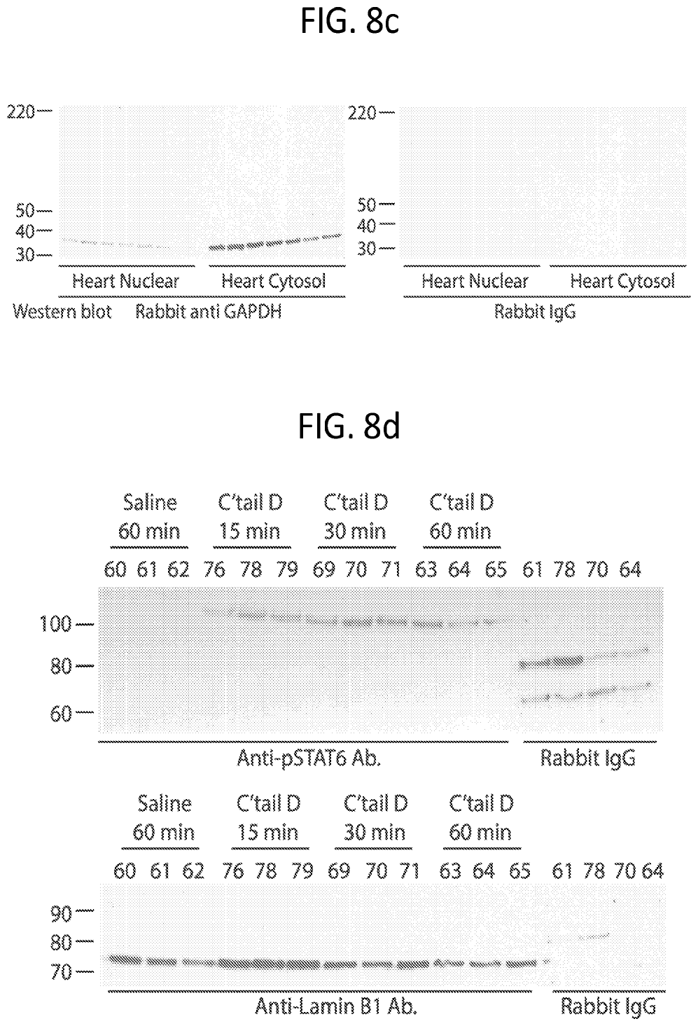

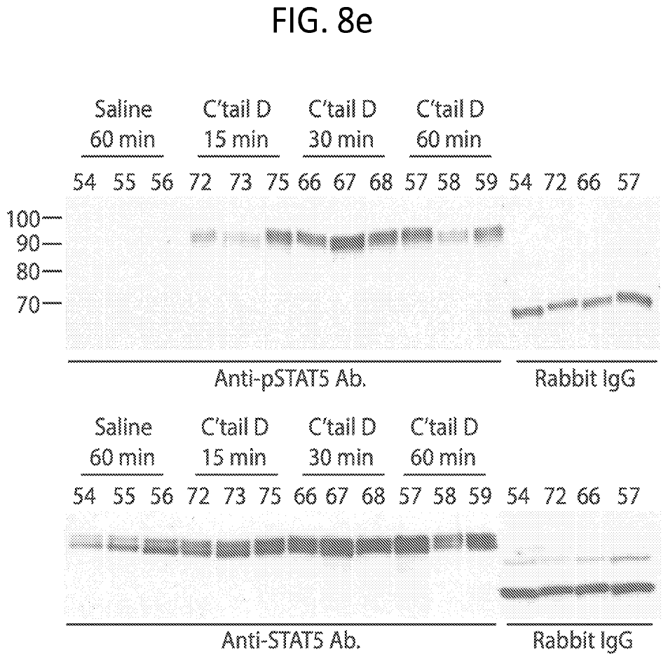

Since ZHX2 hypomorph BALB/cJ mice have much lower mortality than ZHX2 BALB/c mice following induction of the severe cytokine storm model, combination depletion of TNF-α with IL-2 or IL-4 or IL-13 eliminates mortality and reduces morbidity in BALB/c mice, the inventors studied signaling pathways downstream of their receptors. Qualitative studies from whole organ heart, liver and kidney protein extracts confirmed phosphorylation of NFκB pathway component p-p65 (downstream of TNFα receptor), pSTAT5 (downstream of IL-2 receptor), and pSTAT6 (downstream of IL-4 and IL-13 receptor complex) in Cocktail D injected BALB/c and BALB/cJ mice at 15-, 30- and 60-minute time points, but not saline injected controls (examples in FIG. 8a). Next, nuclear and cytosolic proteins were extracted from each organ and quality tested for predominant expression of nuclear protein Lamin B1 in nuclear extracts (examples in FIG. 8b) and GAPDH in both fractions (examples in FIG. 8c). Since IL-2, IL-4 and IL-13 receptors are expressed in the same cells as ZHX2, whereas TNFα receptors are mostly vascular, Western blot and densitometry quantification of nuclear and cytosolic pSTAT5 and pSTAT6 proteins relative to Lamin B1, STAT5 and STAT6 was compared in BALB/c and BALB/cJ mice (FIG. 6a, b, examples of Western blots in FIGS. 8d and 8e). Saline groups did not activate signaling pathways and are not shown in FIG. 6. Higher and/or earlier nuclear pSTAT5 expression was noted in all 3 organs in BALB/cJ compared to BALB/c mice, despite equivalent or lower relative pSTAT5 expression in the cytosolic compartment in most scenarios (FIG. 6a). Overall relative cytosolic pSTAT6 generation was lower in BALB/cJ mice, but relative nuclear pSTAT6 expression was equivalent and in some cases higher in BALB/cJ compared to BALB/c mice (FIG. 6b). These data suggest that pSTAT5 and pSTAT6 move into the nucleus more rapidly and early in ZHX2 deficient BALB/cJ mice compared with BALB/c mice, causing asynchronous activation of target genes in BALB/cJ mice that could prevent more severe injury and higher mortality in BALB/c mice. This means that the nuclear effects of cell signaling pathways in BALB/cJ mice occur too early in the disease process (asynchronous activation), such that other disease pathways in these conditions are not as yet ready for a synergistic effect, as would occur in with appropriate timing of activation in BALB/c mice.

### Example 6

STAT6 Pathway and ZHX Mediated Mechanisms in Cytokine Cocktails Induced glomerular injury

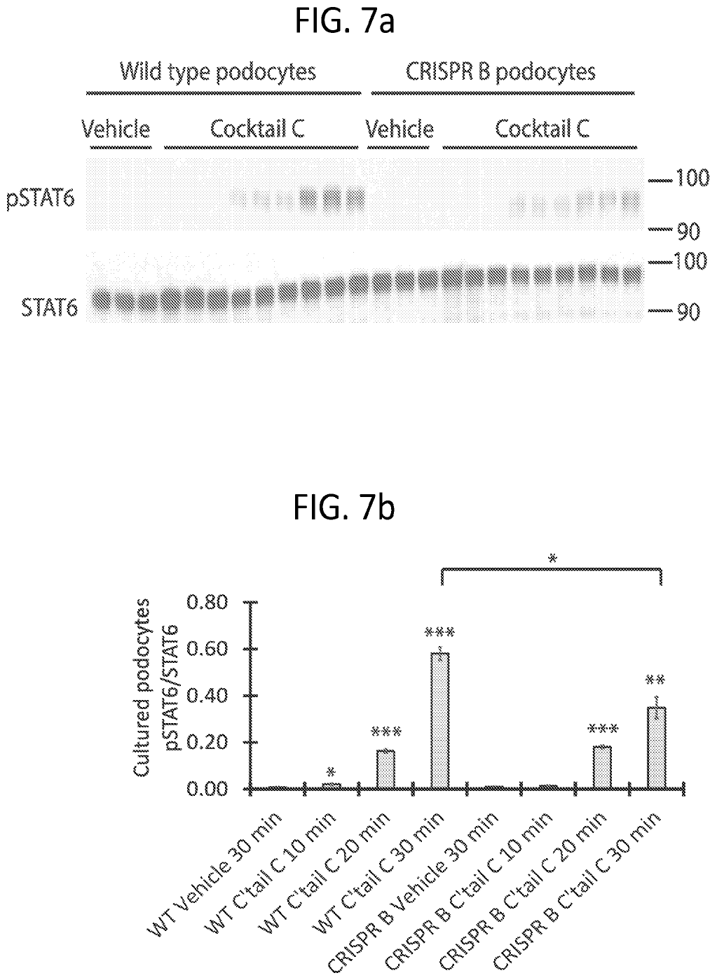

Similar to cytosolic pSTAT6 studies in BALB/cJ mouse liver and kidney (FIG. 6b), Cocktail C induced pSTAT6 phosphorylation was significantly lower at 30 minutes in CRISPR B ZHX2 hypomorph podocytes compared to wild type control (FIG. 7a, b).

(FIG. 7a) Western blots to assess activation of pSTAT6 signaling in wild type and ZHX2 hypomorph (CRISPR B) cultured human podocytes incubated with human counterparts of Cocktail C cocktail (final concentration x/100,000; n=3 plates per condition). (FIG. 7b) Densitometry of Western blot of Cocktail C incubated wild type and CRISPR B podocytes from panel a. *P<0.05; **P<0.01; ***P<0.001, all values based on two-tail analysis.

FIG. 8a-e depicts examples of in vivo signaling mechanisms activated by COVID cocktails injected into mice. (FIG. 8a) Examples of NFκB/p-p65 (liver, 30 minutes), pSTAT6 (kidney 60 minutes) and pSTAT5 (heart, 15 minutes) activation by qualitative Western blot of whole organ protein extracts of mice (n=3 per group) injected with Cocktail D 3× or control saline. (FIG. 8b) Examples of nuclear extract purity studies by Western blot of nuclear and cytosol fractions with equal protein loading from heart, kidney and liver using anti-Lamin B1 antibody. Traces of Lamin B1 in the cytosol may be related to protein synthesis prior to transport to the nucleus. (FIG. 8c) Examples of assessment of GAPDH protein in heart cytosolic and nuclear fractions with equal protein loading from multiple mice. As expected, GAPDH is expressed in both fractions, with greater expression in cytosolic extracts. (FIG. 8d) Example of quantitative Western blots of BALB/c mouse heart nuclear protein extracts (20 μg protein per lane) in a Cocktail D (C′tail D) or control saline injection study assessed for pSTAT6 and Lamin B1 on separate blots developed on the same film. Abbreviated mouse numbers are shown for each lane. (FIG. 8e) Example of quantitative Western blots of BALB/cJ mouse heart cytosolic protein extracts (20 μg protein per lane) in a Cocktail D (C′tail D) or control saline injection study assessed for pSTAT5 and STAT5 on separate blots developed on the same film. Abbreviated mouse numbers are shown for each lane.

### DISCUSSION

The inventors discovered the following:
1) ZHX2 hypomorph BALB/cJ mice show less severe heart, liver and kidney injury compared to ZHX2+/+ mice following exposure to a cytokine cocktail.
2) A genomic basis of the human hypomorph state demonstrating that this deletion at 122,533,694 induces a ZHX2 hypomorph state using CRISPR Cas9 induced modification of human podocyte cell lines. See FIG. 5.
3) In liver, heart and kidney cells, active signaling components of the STAT5 and STAT6 pathways (pSTAT5 and pSTAT6) enter the nucleus earlier and out of synchrony with other molecular events during a cytokine storm in the ZHX2 hypomorph state. See FIG. 6, FIG. 8. This occurs despite overall reduced activation of the STAT6 pathway in the ZHX2 hypomorph state. This asynchronous out-of-turn early activation of target genes produces less severe disease in the ZHX2 hypomorph state.
4) Reduced overall activation of the STAT6 pathway is also noted in human cultured podocytes that have been mutated to replicate 122,533,694 deletion (CRISPR B). See FIG. 7a, b.
5) ZHX2 hypomorph reduces mortality in cytokine storms; when the inventors compared the high dose cytokine storm model in ZHX2+/+ mice (FIG. 4) and ZHX2 hypomorph mice (FIG. 3), it was noted that ZHX2 hypomorph mice have lower mortality (1/6, or 16.6%, FIG. 3, Control IgG group) compared to ZHX2+/+ mice (5/6 or 83.3%; FIG. 4, Control IgG group) under the same conditions.

As will be appreciated from the descriptions herein, a wide variety of aspects and embodiments are contemplated by the present disclosure, examples of which include, without limitation, the aspects and embodiments listed below:

Methods are disclosed in which ZHX2 can be inhibited, neutralized or depleted by the administration of an agent to the patient where the agent comprises an adeno-associated virus (AAV) or lentovirus-containing an a short-hairpin RNA (shRNA) against one ZXH2. The inventors contemplate that the shRNA may be commercially available and can be attached to or part of any vector known in the art including plasmids, viral vectors, bacteriophages, cosmids, and artificial chromosomes.

Methods are disclosed in which ZHX2 can be inhibited, neutralized or depleted by the administration of a polyclonal or a monoclonal antibody directed against the ZXH2.

Methods are disclosed in which ZHX2 can be inhibited, neutralized or depleted by the administration of an siRNA or antisense oligonucleotide that targets ZXH2.

Methods are disclosed in which ZHX2 can be inhibited, neutralized or depleted by the administration of a pharmacological agent that decreases ZHX2 expression.

Methods are disclosed in which ZHX2 can be inhibited, neutralized or depleted by a pharmacological agent that binds or interacts directly or indirectly with the ZHX2 gene, or upstream or downstream of the ZHX2 gene, or makes reversible or irreversible changes at these sites.

It can be contemplated that ZHX2 can be silenced or “turned off” by a pharmacological agent that inhibits or blocks a protein that interacts with ZHX2 in the nucleus. Examples of such proteins include other ZHX proteins, Nuclear Factor Y-A and many others.

The disclosure provides methods of treating various disease states, by inhibiting, blocking or depleting ZHX2 where the disease states include inflammatory and non-inflammatory diseases that affect cytokine release including viral infections like SARS-Cov-1, SARS-Cov-2, other coronaviruses, influenza, parainfluenza, Respiratory Syncytial Virus, adenoviruses, enteroviruses, cytomegalovirus (CMV), Epstein Barr Virus (EBV), Middle East Respiratory Syndrome (MERS), and Ebola, non-respiratory viral infections, non-viral infections like bacterial, fungal and parasitic infections, immune-mediated disorders, cardiovascular pathology, diabetes, metabolic syndrome, organ transplantation, neurodegeneration, and cancer, and aging.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the subject matter described herein may be employed

1. A method for treating a disease in a patient in need thereof comprising the administration of an inhibitor of ZHX2 to the patient, wherein the administration of an inhibitor of ZHX2, wherein the administration of an inhibitor of ZHX2 leads to the depletion of ZHX2 in the patient.

2. The method of claim 1, where in the disease to be treated is selected from the group consisting of inflammatory and non-inflammatory diseases that affect cytokine release including viral infections like SARS-Cov-1, SARS-Cov-2, other coronaviruses, influenza, parainfluenza, Respiratory Syncytial Virus, adenoviruses, enteroviruses, cytomegalovirus (CMV), Epstein Barr Virus (EBV), Middle East Respiratory Syndrome (MERS), and Ebola, non-respiratory viral infections, non-viral infections like bacterial, fungal and parasitic infections, immune-mediated disorders, cardiovascular pathology, diabetes, metabolic syndrome, organ transplantation, neurodegeneration, and cancer, and aging.

3. The method of claim 1, wherein the inhibitor of ZHX2 is selected from the group consisting of agent comprising an adeno-associated virus (AAV) or lentovirus-containing an a short-hairpin RNA (shRNA), an antibody or antibody fragment directed against ZHX2, an siRNA or other antisense oligonucleotide that targets ZHX2, and an antagonist that binds to a ZHX2-mediated receptor.

4. The method of claim 3, wherein shRNA is attached to or part of a vector.

5. The method of claim 4, wherein the vector is selected from the group consisting of plasmids, viral vectors, bacteriophages, cosmids, and artificial chromosomes.

6. The method of claim 3, wherein the antibody or antibody fragment directed against the one or more antibodies selected from the group consisting of a polyclonal antibody, a monoclonal antibody and a bivalent antibody.

7. The method of claim 3, wherein an inhibitor of ZHX2 is one or more DNA fragments encoding a ZHX2 gene that has been modified.

8. The method of claim 7, wherein the one or more DNA fragments encoding a ZHX2 gene that has been modified by CRISPR

9. The method of claim 2, wherein the disease to be treated is a disease that causes an increased release of cytokines.

10. The method of claim 2, wherein the respiratory viral infection is the result of an infection by SARS-COV-2.

11. The method of claim 1, wherein the administration of the ZHX2 inhibitor leads to a ZHX2 hypomorph state in the patient being treated.

12. The method of claim 1, further comprising altered activation of STAT5, STAT6 or NFκB proteins.

13. A method for inhibiting, neutralizing or depleting ZHX2 in a patient in need thereof comprising the administration of a pharmacological agent, wherein the pharmacological agent binds or interacts with the ZHX2 gene and wherein the binding of the pharmacological agent makes reversible or irreversible changes to the ZHX2 gene.

14. The method of claim 13, wherein the pharmacological agent binds or interacts with the ZHX2 gene directly.

15. The method of claim 13, wherein the pharmacological agent binds or interacts with the ZHX2 gene indirectly either upstream or downstream of the ZHX2 gene.

16. A method for reducing or silencing ZHX2 gene expression in a patient in need thereof comprising the administration of a pharmacological agent, wherein the pharmacological agent inhibits or blocks a protein that interacts with ZHX2 in the nucleus of a cell.

17. The method of claim 16, wherein the protein that interacts with ZHX2 in the nucleus is selected from one or more of ZHX1, ZHX3, Nuclear Factor-YA, Nuclear Factor-YB, Nuclear Factor-YC, FoxCI and ephrin-B1/B2 or any other protein known to interact with ZHX2.

18. The method of claim 16, wherein the pharmacological agent that inhibits or blocks a protein that interacts with ZHX2 in the nucleus of a cell causes reversible or irreversible changes to the ZHX2 gene expression.

19. The method of claim 13, wherein the administration of the pharmacological agent leads to a ZHX2 hypomorph state in the patient being treated.

20. The method of claim 16, wherein the administration of the pharmacological agent leads to a ZHX2 hypomorph state in the patient being treated.


SEQUENCE LISTING (37 sequences)

SEQ ID NO: 1 - DNA, 25 bp, synthetic construct
  source 1..25: mol_type = other DNA
caccgactggtaaaccacttagggc

SEQ ID NO: 2 - DNA, 25 bp, synthetic construct
  source 1..25: mol_type = other DNA
aaacgccctaagtggtttaccagtc

SEQ ID NO: 3 - DNA, 28 bp, synthetic construct
  source 1..28: mol_type = other DNA
gcatatacgatacaaggctgttagagag

SEQ ID NO: 4 - DNA, 36 bp, synthetic construct
  source 1..36: mol_type = other DNA
cgggccggatccctagatgtagcattaccagggtgg

SEQ ID NO: 5 - DNA, 41 bp, synthetic construct
  source 1..41: mol_type = other DNA
ggccgaagcttgcagagaagatcacgatagattagaagatg

SEQ ID NO: 6 - DNA, 22 bp, synthetic construct
  source 1..22: mol_type = other DNA
ggtttccttgttatatcaccag

SEQ ID NO: 7 - DNA, 64 bp, synthetic construct
  source 1..64: mol_type = other DNA
gctctaggatgactggtaaaccacttagggcagtcgtccccagacctggtctgtggcctgttag

SEQ ID NO: 8 - DNA, 64 bp, synthetic construct
  source 1..64: mol_type = other DNA
ctaacaggccacagaccaggtctggggacgactgccctaagtggtttaccagtcatcctagagc

SEQ ID NO: 9 - DNA, 45 bp, synthetic construct
  source 1..45: mol_type = other DNA
gattatctttctagggttaacgaacttcaagtaatcaagagcagc

SEQ ID NO: 10 - DNA, 43 bp, synthetic construct
  source 1..43: mol_type = other DNA
cgcagactatctttctagggttaactttgtagaatgcttctcg

SEQ ID NO: 11 - DNA, 43 bp, synthetic construct
  source 1..43: mol_type = other DNA
cgagaagcattctacaaagttaaccctagaaagatagtctgcg

SEQ ID NO: 12 - DNA, 45 bp, synthetic construct
  source 1..45: mol_type = other DNA
gctgctcttgattacttgaagttcgttaaccctagaaagataatc

SEQ ID NO: 13 - DNA, 44 bp, synthetic construct
  source 1..44: mol_type = other DNA
acactgacgacatggttctacagtctctgaaacatagaaggcac

SEQ ID NO: 14 - DNA, 45 bp, synthetic construct
  source 1..45: mol_type = other DNA
tacggtagcagagacttggtctgagaatctaataccgctgatctg

SEQ ID NO: 15 - DNA, 25 bp, synthetic construct
  source 1..25: mol_type = other DNA
caccgacccatccatacacttaccc

SEQ ID NO: 16 - DNA, 25 bp, synthetic construct
  source 1..25: mol_type = other DNA
aaacgggtaagtgtatggatgggtc

SEQ ID NO: 17 - DNA, 28 bp, synthetic construct
  source 1..28: mol_type = other DNA
gcatatacgatacaaggctgttagagag

SEQ ID NO: 18 - DNA, 46 bp, synthetic construct
  source 1..46: mol_type = other DNA
ggcggcactagtctagctggcttgactttacaagacgattccatcc

SEQ ID NO: 19 - DNA, 40 bp, synthetic construct
  source 1..40: mol_type = other DNA
gggcggatccctgcactcagtattctgcaagtcctgtagc

SEQ ID NO: 20 - DNA, 18 bp, synthetic construct
  source 1..18: mol_type = other DNA
cgatctcctgacctcaag

SEQ ID NO: 21 - DNA, 167 bp, synthetic construct
  source 1..167: mol_type = other DNA
gtgcctggcctgttatgatcttcttactcatttgatagcaccagtgtcctgagaaaaataacatatactccattacccatccatacacttacccaggcactcattcaccatattaactagatagacacatgatgttgctgctcctgttgatgataacaatgttgagg

SEQ ID NO: 22 - DNA, 166 bp, synthetic construct
  source 1..166: mol_type = other DNA
cctcaacattgttatcatcaacaggagcagcaacatcatgtgtctatctagttaatatggtgaatgagtgactgggtaagtgtatggatgggtaatggagtatatgttatttttctcaggacactggtgctatcaaatgagtaagaagatcataacaggccaggac

SEQ ID NO: 23 - DNA, 59 bp, synthetic construct
  source 1..59: mol_type = other DNA
cgtcacaatatgattatctttctagggttaactagatagacacatgatgttgctgctcc

SEQ ID NO: 24 - DNA, 56 bp, synthetic construct
  source 1..56: mol_type = other DNA
cgtcaattttacgcagactatctttctagggttaatatggtgaatgagtgactggg

SEQ ID NO: 25 - DNA, 56 bp, synthetic construct
  source 1..56: mol_type = other DNA
cccagtcactcattcaccatattaaccctagaaagatagtctgcgtaaaattgacg

SEQ ID NO: 26 - DNA, 59 bp, synthetic construct
  source 1..59: mol_type = other DNA
ggagcagcaacatcatgtgtctatctagttaaccctagaaagataatcatattgtgacg

SEQ ID NO: 27 - DNA, 60 bp, synthetic construct
  source 1..60: mol_type = other DNA
acactgacgacatggttctacagttatgatcttcttactcatttgatagcaccagtgtcc

SEQ ID NO: 28 - DNA, 52 bp, synthetic construct
  source 1..52: mol_type = other DNA
tacggtagcagagacttggtctgaaaggagcagtgttgatctagagagagcc

SEQ ID NO: 29 - DNA, 21 bp, synthetic construct
  source 1..21: mol_type = other DNA
cggaactggctgaatcagact

SEQ ID NO: 30 - DNA, 24 bp, synthetic construct
  source 1..24: mol_type = other DNA
cagcacagcagttctaacagactt

SEQ ID NO: 31 - DNA, 15 bp, synthetic construct
  source 1..15: mol_type = other DNA
tgcagaggctggcca

SEQ ID NO: 32 - DNA, 10 bp, synthetic construct
  source 1..10: mol_type = other DNA
cacacacaca

SEQ ID NO: 33 - DNA, 20 bp, Homo sapiens
  source 1..20: mol_type = genomic DNA
aatgttgaggtggatggatg

SEQ ID NO: 34 - DNA, 28 bp, synthetic construct
  source 1..28: mol_type = other DNA
aatgttgaggtggatggatggatggatg

SEQ ID NO: 35 - DNA, 20 bp, Homo sapiens
  source 1..20: mol_type = genomic DNA
cgcgcgtgcgcacacacaca

SEQ ID NO: 36 - DNA, 30 bp, Homo sapiens
  source 1..30: mol_type = genomic DNA
cgcgcgtgcacacacacacacacacacaca

SEQ ID NO: 37 - DNA, 20 bp, Homo sapiens
  source 1..20: mol_type = other DNA
cgcgcgtgcacacacacaca